(19) United States
(12) Patent Application Publication
(10) Pub. No.: US 2021/0057061 A1
(43) Pub. Date: Feb. 25, 2021

(54) BIOMETRIC IDENTITY SYSTEM INTEGRATION OF MEDICAL SERVICE PROVIDER SYSTEMS

(71) Applicant: Alclear, LLC, New York, NY (US)

(72) Inventors: Marisa Bass, New York, NY (US); Joe Trelin, New York, NY (US)

(21) Appl. No.: 16/991,121

(22) Filed: Aug. 12, 2020

Related U.S. Application Data
(60) Provisional application No. 62/888,919, filed on Aug. 19, 2019.

(51) Int. Cl. G16H 10/60; G16H 40/20

(52) U.S. Cl. CPC G16H 10/60; G16H 40/20

(57) ABSTRACT

An identity system communicates with different medical service provider systems in order to exchange and/or direct the exchange of information to integrate the systems. The identity system maintains biometric data associated with identities in order to verify the identities for the systems. In various examples, the identity system may receive a request for identity information from a first system and arrange for the identity information to be provided to the first system and a second system upon determining that the request is authorized. In some examples, the identity system may receive an update to the identity information from the first system and arrange for the update to be provided to the second system. In a number of examples, the identity system may determine that the first system stores identity information that is missing from the second system and may arrange for the identity information to be provided to the second system.

### CROSS-REFERENCE TO RELATED APPLICATION(S)

This application is a nonprovisional patent application of and claims the benefit of U.S. Patent Application No. 62/888,919, filed Aug. 19, 2019 and titled “Biometric Identity System Integration of Medical Service Provider Systems,” the disclosure of which is hereby incorporated herein by reference in its entirety.

### FIELD

The described embodiments relate generally to biometric identity systems. More particularly, the present embodiments relate to biometric identity system integration of medical service provider systems.

### BACKGROUND

Entities, such as a medical service provider or other enterprise, may have a number of different systems. For example, a medical service provider may have an accounting system, a scheduling system, a medical records system, a payment system, and so on. The different systems may be configured to handle different operations that an entity performs. For example, office staff at a medical service provider may use a scheduling system to manage patient appointments whereas doctors at a medical service provider may use a medical records system to track patient care.

Some such systems may use biometrics to identify people. Use of biometrics to establish a person's identity is increasingly common. Biometrics are used to unlock electronic devices, retrieve sensitive information, enter secure areas (such as prescription drug storage areas, as one example), rent or purchase goods, and so on. Some common biometric reader devices involve physical contact by a user, as is the case with most fingerprint readers. Other biometric reader devices require that a user stand relatively close to the device, as with an iris scanner or face recognition device.

### SUMMARY

The present disclosure relates to biometric identity system integration of medical service provider systems. An identity system may communicate with different medical service provider systems in order to exchange and/or direct the exchange of information. The identity system may maintain biometric data associated with identities in order to verify the identities for the medical service provider systems. In various examples, the identity system may receive a request for identity information from a first system and arrange for the identity information to be provided to the first system and a second system upon determining that the request is authorized. In some examples, the identity system may receive an update to the identity information from the first system and arrange for the update to be provided to the second system. In a number of examples, the identity system may determine that the first system stores identity information that is missing from the second system and may arrange for the identity information to be provided to the second system.

In various embodiments, a system for biometric identity system integration of medical service provider systems includes at least one non-transitory storage medium that stores instructions and at least one processor. The at least one processor executes the instructions to maintain biometric data associated with identities in order to verify the identities for a first medical service provider system device and a second medical service provider system device; receive a request for identity information associated with an identity from the first medical service provider system device; and upon determining that the request is authorized, arrange for the identity information to be provided to the first medical service provider system device and the second medical service provider system device.

In some examples, the request includes a digital representation of a biometric. In various implementations of such examples, the identity is a first identity and the at least one processor determines that the request is authorized when the digital representation of the biometric matches first biometric data associated with the first identity or matches second biometric data associated with a second identity that has permission to access the identity information.

In a number of examples, the first medical service provider system device and the second medical service provider system device comprise devices in at least two of an accounting system, a scheduling system, a calendaring system, a payment system, a medical record system, a check in system, a building access system, an insurance system, a medical procedure authorization system, and a medication access system. In various examples, the at least one processor arranges for the identity information to be provided to the first medical service provider system device and the second medical service provider system device by transmitting the identity information to the first medical service provider system device and the second medical service provider system device. In some examples, the at least one processor arranges for the identity information to be provided to the first medical service provider system device and the second medical service provider system device by instructing an electronic device to transmit the identity information to the first medical service provider system device and the second medical service provider system device. In a number or examples, the at least one processor determines that the second medical service provider system device is authorized to receive the identity information before arranging for the identity information to be provided to the second medical service provider system device.

In some embodiments, a system for biometric identity system integration of medical service provider systems includes at least one non-transitory storage medium that stores instructions and at least one processor. The at least one processor executes the instructions to maintain biometric data associated with identities in order to verify the identities for a first medical service provider system device and a second medical service provider system device, receive an update to identity information associated with an identity from the first medical service provider system device, and arrange for the update to be provided to the second medical service provider system device.

In various examples, the update is at least one of a name, an address, a medical record, insurance information, or payment information. In some examples, the update is received from a person corresponding to the identity. In a number of examples, the identity is a first identity and the update is received from a person corresponding to a second identity that is authorized to modify the identity information.

In some examples, the at least one processor determines that the update is authorized prior to arranging for the update to be provided to the second medical service provider system device. In a number of implementations of such examples, the at least one processor updates the identity information upon determining that the update is authorized. In various implementations of such examples, the at least one processor determines that the update is authorized by comparing a received digital representation of a biometric to respective biometric data associated with the identity.

In a number of embodiments, a system for biometric identity system integration of medical service provider systems includes at least one non-transitory storage medium that stores instructions and at least one processor. The at least one processor executes the instructions to maintain biometric data associated with identities in order to verify the identities for a first medical service provider system device and a second medical service provider system device, determine that the first medical service provider system device stores identity information that is associated with an identity and missing from the second medical service provider system device, and arrange for the identity information to be provided to the second medical service provider system device.

In some examples, the at least one processor receives an identity verification request including a digital representation of a biometric and provides a response to the identity verification request based on a comparison of the digital representation of the biometric to at least a portion of the biometric data. In a number of implementations of such examples, the response identifies a respective identity that is associated with respective biometric data that matches the digital representation of the biometric.

In various examples, the at least one processor determines that the identity information is covered by a specification listing information to be duplicated on the first medical service provider system device and the second medical service provider system device. In some examples, the at least one processor determines that the second medical service provider system device is covered by a specification listing device on which to duplicate information from the first medical service provider system device. In a number of examples, the first medical service provider system device is not configured to transmit the identity information directly to the second medical service provider system device absent involvement of the at least one processor.

### BRIEF DESCRIPTION OF THE DRAWINGS

The disclosure will be readily understood by the following detailed description in conjunction with the accompanying drawings, wherein like reference numerals designate like structural elements.

FIG. 1 depicts a first example system for biometric secured medical check in.

FIG. 2 is a flow chart illustrating a first example method for biometric secured medical check in. This method may be performed by the system of FIG. 1.

FIG. 3 depicts a second example system for biometric secured medical check in.

FIG. 4 is a flow chart illustrating a second example method for biometric secured medical check in. This method may be performed by the systems of FIGS. 1 and/or 3.

FIG. 5 is a flow chart illustrating a third example method for biometric secured medical check in. This method may be performed by the systems of FIGS. 1 and/or 3.

FIG. 6A depicts a third example system for biometric secured medical check in.

FIG. 6B depicts the system of FIG. 6A upon check in.

FIG. 7 is a flow chart illustrating a third example method for biometric secured medical check in. This method may be performed by the systems of FIGS. 1, 3, and/or 6A and 6B.

FIG. 8A depicts a fourth example system for biometric secured medical check in.

FIG. 8B depicts the system of FIG. 8A upon check in.

FIG. 9 is a flow chart illustrating a fourth example method for biometric secured medical check in. This method may be performed by the systems of FIGS. 1, 3, and/or 8A and 8B.

FIG. 10A depicts a fifth example system for biometric secured medical check in.

FIG. 10B depicts the system of FIG. 10A as medications are provided.

FIG. 11 is a flow chart illustrating a fifth example method for biometric secured medical check in. This method may be performed by the systems of FIGS. 1, 3, and/or 10A and 10B.

FIG. 12A depicts a sixth example system for biometric secured medical check in.

FIG. 12B depicts the system of FIG. 12A upon vending.

FIG. 13 is a flow chart illustrating a fifth example method for biometric secured medical check in. This method may be performed by the systems of FIGS. 1, 3, and/or 12A and 12B.

FIG. 14 depicts a first example system for biometric identity system integration of medical service provider systems.

FIG. 15 depicts a second example system for biometric identity system integration of medical service provider systems.

FIG. 16 is a flow chart illustrating a first example method for biometric identity system integration of medical service provider systems. This method may be performed by the systems of FIGS. 1, 3, 14, and/or 15.

FIG. 17 is a flow chart illustrating a second example method for biometric identity system integration of medical service provider systems. This method may be performed by the systems of FIGS. 1, 3, 14, and/or 15.

FIG. 18 is a flow chart illustrating a third example method for biometric identity system integration of medical service provider systems. This method may be performed by the systems of FIGS. 1, 3, 14, and/or 15.

FIG. 19 is a flow chart illustrating a fourth example method for biometric identity system integration of medical service provider systems. This method may be performed by the systems of FIGS. 1, 3, 14, and/or 15.

FIG. 20 is a flow chart illustrating a fifth example method for biometric identity system integration of medical service provider systems. This method may be performed by the systems of FIGS. 1, 3, 14, and/or 15.

FIG. 21 is a flow chart illustrating a sixth example method for biometric identity system integration of medical service provider systems. This method may be performed by the systems of FIGS. 1, 3, 14, and/or 15.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, apparatuses, methods, and computer program products that embody various elements of the present disclosure. However, it should be understood that the described disclosure may be practiced in a variety of forms in addition to those described herein.

Different systems used by an entity, such as a medical service provider or other enterprise, may not be configured to communicate with each other. In some situations, the different systems may be configured to communicate with each other, but may not be configured to exchange information that is relevant to each of the different systems. Some of the systems may even store information that is relevant to other systems in a hardcopy or other analog format that is not capable of being shared without scanning, data entry, or other conversion.

For example, staff at a reception desk may check a patient in for an appointment using a scheduling system. As part of this process, the staff may have the person fill out a form with their name, address, telephone number, and other information. The staff may also photocopy the person's identification, insurance card, and so on. These forms and photocopies may be stored in a physical filing system associated with the scheduling system. Subsequently, a doctor may use a medical records system as part of providing one or more medical services to the person. The medical records system may not be capable of accessing any of the information from the physical filing system because it is stored in hardcopy instead of electronically. The scheduling system and the medical records system may not even be configured to exchange electronic information that they do store and both use. As a result, useful data may be lacking in one or more of the systems, redundant and/or out of date data may be entered and/or stored, redundant components may be required for the systems for such redundant data to be entered and/or stored, and so on. The systems may not be efficient due to the unnecessary data entry, lack of access to data and/or up to date data, and so on.

The systems may be configured to use an identity system for biometric identification. For example, each of the systems may be configured to provide captured biometric data to an identity system that may identify a person using the biometric data and provide one or more attestations (such as an identity of the person, identity information associated with the identity stored by the identity system and/or another system for which the identity system controls and/or directs data access, confirmations regarding identity information like a verification that a person is of a minimum age or has insurance coverage, and so on). In some situations, the systems may use the identity system to verify the identity of a patient, obtain identity information and/or confirmations about identity information for the patient, and so on. In other situations, the systems may use the identity system to verify the identity of an employee (such as a doctor, nurse, support staff, and so on), obtain identity information and/or confirmations about identity information for the employee (such as an employee's access rights to a patient's medical records, an employee's medical certifications and/or qualifications, and so on), and so on.

As the systems each communicate with the identity system, the identity system may be used to integrate the different systems. The identity system may communicate with the different systems to provide information stored by the identity system and/or by other systems for which the identity system is able to control and/or direct data access. In this way, the different systems may have the enhanced functions of access to and/or of exchange data that they would not otherwise be able to perform. This may improve operation of the systems as useful data may not be lacking in one or more of the systems, redundant and/or out of date data may be eliminated and/or reduced, redundant components may be eliminated that would otherwise be used for such redundant data to be entered and/or stored, and so on. The systems may be more efficient due to reduced or eliminated unnecessary data entry, reduced or eliminated lack of access to data and/or up to date data, and so on.

The present disclosure relates to biometric identity system integration of medical service provider systems. An identity system may communicate with different medical service provider systems in order to exchange and/or direct the exchange of information. The identity system may maintain biometric data associated with identities in order to verify the identities for the medical service provider systems. In various examples, the identity system may receive a request for identity information from a first system and arrange for the identity information to be provided to the first system and a second system upon determining that the request is authorized. In some examples, the identity system may receive an update to the identity information from the first system and arrange for the update to be provided to the second system. In a number of examples, the identity system may determine that the first system stores identity information that is missing from the second system and may arrange for the identity information to be provided to the second system.

These and other embodiments are discussed and elaborated further below. Various configurations are possible and contemplated.

In other embodiments, the present disclosure may relate to a system for biometric secured medical check in. Typical check in procedures for medical services are often burdensome, time consuming, and highly inefficient. People may find it inconvenient to fill out check in forms. Such forms may be lengthy, particularly when standardized to cover as many different patient situations as possible, and people may be required to fill out information that they have previously provided to other medical service providers and/or do not have currently available (such as when people do not have a copy of their full medical history or medical list on them when checking in, cannot remember vaccination dates, and so on). These kinds of procedures also involve personnel to provide the forms and/or otherwise obtain the information from the people, interpret the provided information and/or otherwise enter such information into various electronic systems, match information to appointments and/or schedule medical services if there is no appointment, charge insurance and/or payment accounts, calculate copays, and a variety of other tasks. Several of these issues may result in delays, burdens, and/or other inefficiencies, as well as failure to obtain useful information (such as insurance coverage, copays, and/or other payments that may not be collectible later, allergies that may cause complications during the medical services like latex allergies, and so on).

The present disclosure may make check in procedures less burdensome, time consuming, and inefficient by storing identity information for people that may be retrieved upon check in. However, some implementations of such an approach may use a great deal of storage and/or other electronic components in situations where each medical service provider stores the information. Such implementations may still involve people providing a great deal of duplicate information to different medical service providers. In other implementations of such an approach, identity information could be stored in a centrally accessible location that different medical service providers could access. However, such a solution could make it difficult for people to ensure that they have control over access to their identity information.

In some implementations, one or more biometrics could be used to control access to identity information. In such an implementation, people could provide the biometric to enable access and retrieval of the identity information. This may allow the person to check in by providing the biometric without specifying additional information.

However, biometrically securing access to centrally stored identity information may present other issues. The system that uses the biometrics to centrally control access to identity information may be configured to expect inputs (such as one or more digital representations of biometrics, requests for specific identity information and/or attestations regarding specific identity information, and so on) in a particular format. This may be solved by using identical electronic devices at all medical service provider locations so that biometrics and requests involving such are all submitted in an expected way, but this is not particularly flexible.

However, the present disclosure may resolve such issues by using client apps or applications that may run on a variety of different hardware but all submit digital representations of biometrics and related requests in a uniform data structure format. In this way, a system that uses the biometrics to centrally (and/or virtually centrally in implementations where the system is implemented as used within a cloud network) control access to identity information may be configured to process the uniform data structure format to receive, extract, process, and respond to any digital representation of any biometrics and/or related requests regardless of the hardware used to obtain and/or transmit a digital representation of a biometric and/or a related request, the medical service provider who implements and/or uses such hardware, and so on. Further, such a uniform data structure format may allow the system to use different biometrics, different numbers of biometrics, and/or respond to different requests without reconfiguration of the system, client apps or applications, hardware used to obtain and/or transmit digital representations of biometrics and/or related requests, and so on.

In this way, systems described by the present disclosure may be able to check in people for a variety of different medical services at a variety of different medical service providers in a way that is not burdensome while being efficient. Additionally, such systems may be able to perform functions not possible by previous systems while reducing duplicated components, reducing excess processing, reducing excess communication network traffic, improving the efficiency of system hardware and software resources, reducing the number of personnel used to operate the system, and so on.

As such, the following disclosure may also relate to a system for biometric secured medical check in. The system may receive one or more digital representations of biometrics for a person, use the digital representation of the biometric to retrieve identity information for the person, and provide the identity information to a medical service electronic device to check in the person for a medical service. In some implementations, the system may use the digital representation of the biometric to retrieve a medical record identifier for the person and facilitate access to a medical record for the person stored by a medical records electronic device. In various implementations, the system may process payment for the medical service using payment information stored in association with the identity information. In a number of implementations, the system may receive the digital representation of the biometric from a check in electronic device and provide an acknowledgement based on a response received from the medical service electronic device to the check in electronic device.

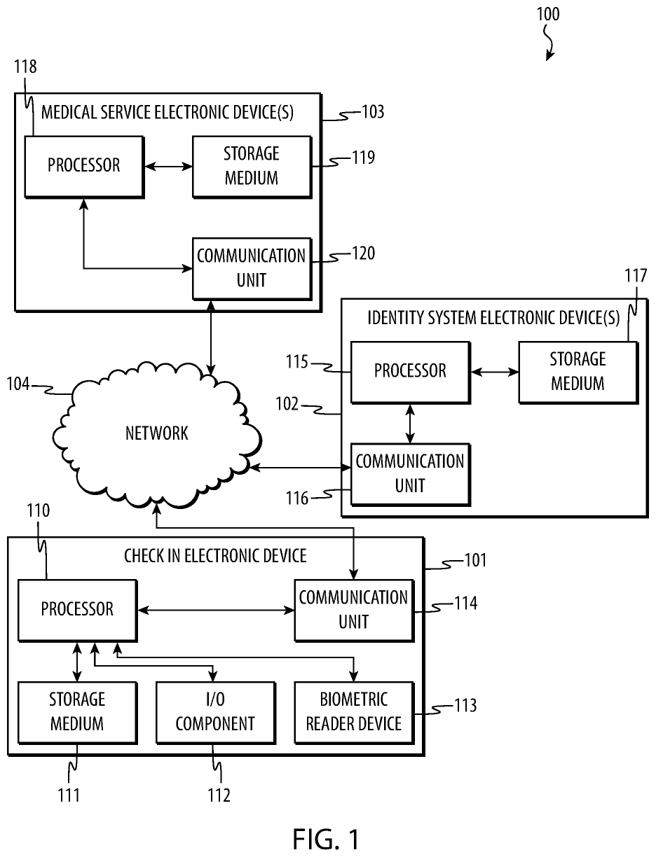

These and other embodiments are discussed below with reference to FIGS. 1-21. However, those skilled in the art will readily appreciate that the detailed description given herein with respect to these Figures is for explanatory purposes only and should not be construed as limiting.

FIG. 1 depicts a first example system 100 for biometric secured medical check in. The system 100 may include a check in electronic device 101, one or more identity system electronic devices 102, and/or one or more medical service electronic devices 103 that may be operative to communicate with each other via one or more communication networks 104.

The check in electronic device 101 may obtain one or more digital representations (which may be in the form of one or more hashes of an electronic representation of the biometric and/or other data structures) of one or more biometrics from a person. The check in electronic device 101 may provide the digital representation of the biometric to the identity system electronic device 102. Alternatively, the check in electronic device 101 may provide the digital representation of the biometric to the identity system electronic device 102 via the medical service electronic device 103. The identity system electronic device 102 may receive the digital representation of the biometric, use the digital representation of the biometric to retrieve one or more sets of identity information associated with the person, and provide the retrieved identity information to the medical service electronic device 103. The medical service electronic device 103 may receive the identity information and use the identity information to check in the person for a medical service.

For example, a person may provide a fingerprint, facial image, and/or other biometric to the check in electronic device 101. The check in electronic device 101 may transmit a digital representation of the biometric to the identity system electronic device 102, which may use the digital representation of the biometric to retrieve a name and/or other patient identifier for the person and provide the name and/or other patient identifier for the person to the medical service electronic device 103. The medical service electronic device 103 may use the name to determine that the person has an appointment for a medical service and check in the person for that determined medical service.

In various implementations, the identity system electronic device 102 and/or the medical service electronic device 103 may provide one more responses and/or acknowledgements to the person via the check in electronic device 101. For example, the check in electronic device 101 may present one or more check in confirmations based on a received response and/or acknowledgment. Such a check in confirmation may include directions regarding where to go (such as a room number) for the medical service, instructions regarding preparation for the medical service (such as instructions regarding rolling up a sleeve in preparation for an inoculation), an estimated wait time, information regarding the medical service that is to be provided (such as a description of a procedure, information regarding a medical professional who will provide the medical service, and so on), information regarding future medical services to be provided and/or scheduled, and so on, costs associated with the medical service, and so on. By way of another example, the check in electronic device 101 may present one or more prompts based on a received response and/or acknowledgment. Such prompts may include a request for insurance information, payment account information, authorization to release identity information and/or medical records, directions regarding specific sets of identity information and/or medical records to release, selection of a medical service provider location, selection between a number of possible medical service appointments, medical waiver signature, acknowledgement of medical service risks, and so on. Alternatively, in other implementations, such check in confirmations, prompts, and so on may be transmitted to an electronic device associated with the person instead of the check in electronic device 101.

In some implementations, the identity system electronic device 102 may determine the medical service electronic device 103 to which to provide the identity information. For example, the identity system electronic device 102 may receive location information from the check in electronic device 101 (such as location information provided and/or selected by the person, included in an identifier provided by the check in electronic device 101, such as a network address, obtained via a global positioning system device, and so on) and determine the medical service electronic device 103 that corresponds to that location.

In various implementations, the identity system electronic device 102 may allow the person to control access to the identity information and/or other information (such as payment account information, medical records, Health Insurance Portability and Accountability Act protected information in order to be compliant with various legal restrictions, and so on). The identity system electronic device 102 may control access to such information according to input received from the person.

The system 100 may protect data by avoiding storing identity information and/or biometric data and/or other information at the medical service electronic device 103 and/or the check in electronic device 101. The system 100 may also protect data by using biometrics to control access to the devices that do store such data. In other implementations, the system 100 may perform other functions, such as charging insurance for medical services, charging a payment account for a medical service, communicating with a medical record database to facilitate secure access by the medical service electronic device 103 to medical records, providing expedited and/or discounted access to medical services and/or complimentary and/or other goods or services to premium account holders or reward account holders, contacting a person's preferred pharmacy to provide a prescription for the person, contacting pharmacies to evaluate costs and/or compare costs between a person's preferred pharmacy and at least one other pharmacy, offering a person to send a prescription to a cheaper pharmacy than the person's preferred pharmacy and/or informing the person of the cost savings, and so on. Various configurations are possible and contemplated without departing from the scope of the present disclosure.

The check in electronic device 101 may be any kind of device. The check in electronic device 101 may be provided by the medical service provider (such as a kiosk or other station in a reception area), may be a device associated with the person (such as the person's mobile telephone), and so on. Examples of such devices include, but are not limited to, one or more desktop computing devices, laptop computing devices, mobile computing devices, wearable devices, tablet computing devices, mobile telephones, smart phones, printers, displays, vehicles, kitchen appliances, entertainment system devices, digital media players, and so on. The check in electronic device 101 may include one or more processing units 110 and/or other processors and/or controllers, one or more non-transitory storage media 111 (which may take the form of, but is not limited to, a magnetic storage medium; optical storage medium; magneto-optical storage medium; read only memory; random access memory; erasable programmable memory; flash memory; and so on), one or more input/output components 112 (such as one or more displays, touch screens, printers, microphones, speakers, keyboards, computer mice, track pads, and so on), one or more biometric reader devices 113 (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on), one or more communication units 114, and/or one or more other components. The processing unit 110 may execute one or more sets of instructions stored in the non-transitory storage media 111 to perform various functions, such as using the biometric reader device 113 to obtain one or more digital representations of one or more biometrics (such as a digital representation of a fingerprint, a blood vessel scan, a palm-vein scan, a voiceprint, a facial image, a retina image, an iris image, a deoxyribonucleic acid sequence, a heart rhythm, a gait, and so on) for a person, communicate with the identity system electronic device 102 and/or the medical service electronic device 103 via the network 104 using the communication unit 114, and so on.

Similarly, the identity system electronic device 102 may be any kind of electronic device and/or cloud and/or other computing arrangement and may include one or more processing units 115, communication units 116, non-transitory storage media 117, and/or other components. The processing unit 115 may execute one or more sets of instructions stored in the non-transitory storage media 117 to perform various functions, such as storing biometric data (which may include one or more digital representations of one or more fingerprints, blood vessel scans, palm-vein scans, voiceprints, facial images, retina images, iris images, deoxyribonucleic acid sequences, heart rhythms, gaits, and so on) for people and associated identity information (such as one or more names, addresses, telephone numbers, social security numbers, patient identification number or other identifier, insurance data, financial data, medical history, and so on), receiving one or more digital representations of biometrics, matching one or more received digital representations of biometrics to stored biometric data, retrieving identity information associated with stored biometric data matching one or more received digital representations of biometrics, providing retrieved identity information, communicating with the check in electronic device 101 and/or the medical service electronic device 103 via the network 104 using the communication unit 116, and so on.

Likewise, the medical service electronic device 103 may be any kind of electronic device and/or cloud and/or other computing arrangement and may include one or more processing units 118, non-transitory storage media 119, communication units 120, and/or other components. The processing unit 118 may execute one or more sets of instructions stored in the non-transitory storage media 119 to perform various functions, such as store information regarding one or more medical services and/or appointments for one or more medical services, receive identity information, check in people for medical services using received identity information, communicate with the check in electronic device 101 and/or identity system electronic device 102 via the network 104 using the communication unit 120, and so on.

Although the system 100 is illustrated and described as including particular components arranged in a particular configuration that perform particular functions, it is understood that this is an example. In various implementations, various arrangements of various components that perform various functions may be implemented without departing from the scope of the present disclosure.

For example, in some implementations, the functions of the check in electronic device 101 may be performed using an app or application (such as an Internet browser) executing on a person's portable computing device (such as a smart phone) and the functions of the medical service electronic device 103 may be performed by a networked group of computing devices located at a medical service provider's location. However, in other implementations, a single electronic device or group of devices may perform the functions of both the check in electronic device 101 and the medical service electronic device 103. For example, a kiosk or other station located in the reception area of a medical service provider's office may be operative to receive digital representations of biometrics, transmit the digital representations of biometrics to the identity system electronic device 102, receive identity information, determine an appointment corresponding to information included in the identity information, check in a person for a determined appointment using the identity information, and/or various other functions. Various configurations are possible and contemplated without departing from the scope of the present disclosure.

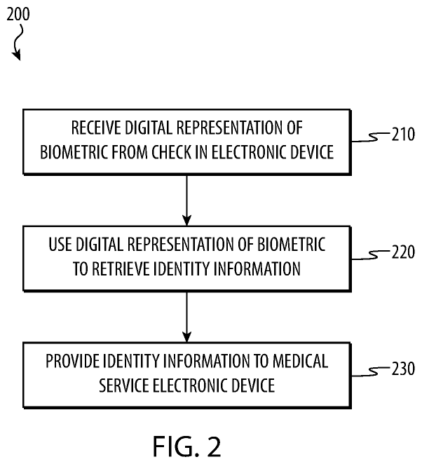

FIG. 2 is a flow chart illustrating a first example method 200 for biometric secured medical check in. This method 200 may be performed by the system 100 of FIG. 1.

At operation 210, an electronic device, such as the identity system electronic device 102, may receive a digital representation of a biometric. The electronic device may receive the digital representation of the biometric from a check in electronic device.

At operation 220, the electronic device may use the digital representation of the biometric to retrieve identity information. For example, the electronic device may match the digital representation of the biometric to stored biometric data and retrieve identity information (whether stored by the electronic device or another device) that is associated with matching stored biometric data.

At operation 230, the electronic device may provide the retrieved identity information to a medical service electronic device. For example, the electronic device may transmit a retrieved patient identification number and/or other patient identifier to a medical service electronic device to facilitate checking in a person for a medical service appointment.

In various examples, this example method 200 may be implemented as a group of interrelated software modules or components that perform various functions discussed herein. These software modules or components may be executed within a cloud network and/or by one or more computing devices, such as the identity system electronic device 102, medical service electronic device 103, and/or the check in electronic device 101 of FIG. 1.

Although the example method 200 is illustrated and described as including particular operations performed in a particular order, it is understood that this is an example. In various implementations, various orders of the same, similar, and/or different operations may be performed without departing from the scope of the present disclosure.

For example, the method 200 is illustrated and described as retrieving and providing identity information. However, in some implementations, only a subset of retrieved identity information may be provided. For example, the electronic device may retrieve more identity information than is requested and may only provide the requested identity information. In other examples, the electronic device may receive more identity information than the electronic device is authorized to provide and may only provide the authorized identity information. Various configurations are possible and contemplated without departing from the scope of the present disclosure.

In a number of implementations, a system for biometric secured medical check in may include at least one non-transitory storage medium that stores instructions and at least one processor. The at least one processor may execute the instructions to receive a digital representation of a biometric of a person from a check in electronic device, use the digital representation of the biometric to retrieve identity information for the person, check the person in for a medical service by providing the identity information to a medical service electronic device, receive a response from the medical service electronic device, and provide an acknowledgment based on the response to the check in electronic device.

In some examples, the acknowledgement may prompt for authorization to access a medical record for the person. In various examples, the acknowledgement may include an instruction regarding a location to report to receive the medical service. In a number of examples, the at least one processor may determine the medical service electronic device to provide the identity information using location information provided via the check in electronic device. In some examples, the at least one processor may determine the medical service electronic device to provide the identity information using a location of the check in electronic device. In various examples, the system may transmit a reminder prior to check in, such as a check in reminder corresponding to an appointment that is transmitted to a remote or other mobile device that enables a person to check in remotely ahead of time and then confirm check in upon arrival. Such a check in reminder may include a notification for a potential charge for a missed appointment, offer incentives for checking in ahead of time (such as a discounted copay in order to reward people in order for medical service providers to have a better idea what their schedule for the day will be, and so on).

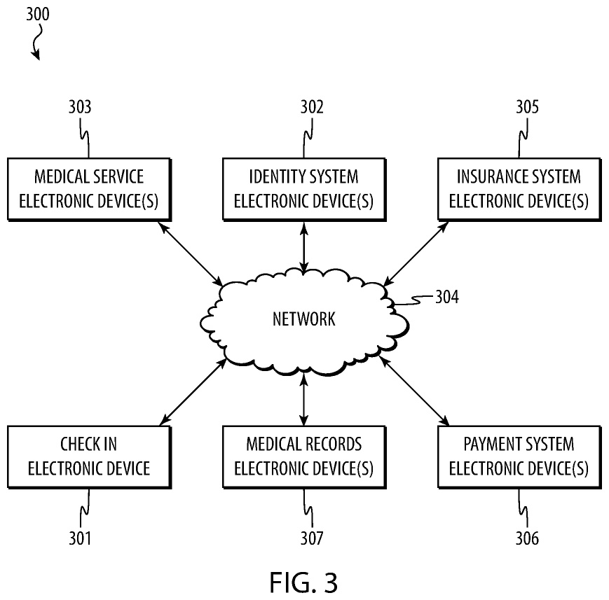

FIG. 3 depicts a second example system 300 for biometric secured medical check in. The system 300 may include a check in electronic device 301, one or more identity system electronic devices 302, one or more medical service electronic devices 303, one or more insurance system electronic devices 305 (such as an insurance claim system electronic device, an insurance coverage verification and/or information electronic device, and so on), one or more payment system electronic devices 306 (such as a credit card and/or other payment processing system electronic device), and/or one or more medical records electronic devices 307 (such as a database to securely store medical records and/or other electronic device) that may be operative to communicate with each other via one or more communication networks 304.

Similarly to the system 100 of FIG. 1, the identity system electronic device 302 may be operable to receive one or more digital representations of biometrics, use the digital representation of the biometric to retrieve identity information, and provide the identity information to the medical service electronic device 303 and/or one or more other electronic devices. Additionally, the identity system electronic device 302 may be operable to process payments and/or facilitate payment processing for one or more medical services.

For example, the identity system electronic device 302 may store insurance information included in and/or associated with the identity information. The identity system electronic device 302 may receive information from the medical service electronic device 303 regarding a medical service, the cost of a medical service, and so on; retrieve the insurance information; and communicate with the insurance system electronic device 305 to charge the insurance and/or otherwise process payment for the medical service. Alternatively, the identity system electronic device 302 may provide the insurance information to the medical service electronic device 303 and the medical service electronic device 303 may communicate with the insurance system electronic device 305 directly.

By way of another example, the identity system electronic device 302 may store financial information (such as one or more credit card numbers, health savings account numbers, flex spending account numbers, debit card numbers, checking or savings account numbers, and/or other financial account numbers, such as an airline mileage account or other rewards or loyalty account that may be used to make a payment) included in and/or associated with the identity information. The identity system electronic device 302 may receive information from the medical service electronic device 303 and/or the insurance system electronic device 305 regarding a medical service, the cost of a medical service, a copay or other payment amount a person is responsible for despite insurance coverage, and so on; retrieve the financial information; and communicate with the payment system electronic device 306 to process a payment, facilitate payment processing, obtain payment for a copay, charge a financial account number, and so on. Alternatively, the identity system electronic device 302 may provide the financial account number and/or other financial and/or payment information (such as insurance information) to the medical service electronic device 303 and the medical service electronic device 303 may communicate with the payment system electronic device 306 directly.

Moreover, the identity system electronic device 302 may be operable to facilitate and/or otherwise provide access to one or more medical records, such as those stored by the medical records electronic device 307. For example, the identity system electronic device 302 may store one or more medical record identifiers in and/or otherwise associated with the identity information. By way of another example, the medical record identifier may be based on the identity information, such as an implementation where the medical record identifier is a hash of at least a portion of the identity information (such as a hash of a digital representation of a biometric, a hash of a social security number or other identifier that can be used to uniquely identify a person without providing access to the identifier, a hash of a name and a social security number, and so on) coupled with an identifier for one of a number of different records repositories such that the medical record identifier is a unique medical record identifier enabling access into that specific records repository. In various examples, the identity information may include data enabling translation between a patient identifier used by an individual medical service provider and/or group of medical service providers and the medical record identifier. Regardless, the identity system electronic device 302 may be operative to retrieve the medical record identifier and use the medical record identifier to facilitate access by the medical service electronic device 303 to a medical record stored by the medical records electronic device 307. In this way, the identity system electronic device 302 may facilitate access to the medical records without storing the medical records.

For example, the identity system electronic device 302 may provide the medical record identifier and/or a specification of medical records requested to the medical records electronic device 307, receive and then provide one or more medical records (such as to the medical service electronic device 303) and/or direct where such medical records should be received, and so on. In some implementations, the identity system electronic device 302 may obtain authorization first, such as by communicating with the check in electronic device 301 and/or another device associated with the person to obtain authorization to access medical records, by referencing stored preferences regarding medical record access, and so on. By way of another example, the identity system electronic device 302 may provide the medical record identifier to another device (such as the medical service electronic device 303) that may then communicate with the medical records electronic device 307 directly.

The medical records electronic device 307 may centrally store medical records (and/or virtually centrally store in implementations where the medical records electronic device 307 is implemented as used within a cloud network) for a person from a variety of different medical service providers. As such, a person may not be required to remember and/or bring complex medical history information and/or go through burdensome and/or time consuming procedures to transfer medical records (such as when switching doctors). The medical records electronic device 307 may be configured to receive updates regarding provided medical services and/or other information to store in medical records (such as information from the medical service electronic device 303 regarding a medical service that is provided to a person), provide medical records in response to receiving a medical record identifier and/or legal authorization to provide medical records, push updates to associated authorized medical service providers, provide notifications to associated authorized medical service providers that updates are available for them to access, and so on. The medical records electronic device 307 may include one or more medical histories and/or portions thereof, allergies, vaccination lists, electronic health record data, electronic medical record data, patient chart data, Health Insurance Portability and Accountability Act and/or other consent forms, current medication lists, prescriptions, previous surgeries, previous hospitalizations, medical consents, upcoming medical service appointments, lab diagnostic results, lab imaging results, order for lab tests, and/or any other medical record information.

Additionally, in various implementations, the identity system electronic device 302 may be operable to provide one or more attestations regarding a person associated with the identity information. For example, a medical service may be restricted to people who are at least 21 years of age and/or who have parental consent. In such implementations, the identity system electronic device 302 may store a verified age for the person (such as in the identity information and/or associated therewith) and/or be operable to communicate with an age verification database. As such, the identity system electronic device 302 may be operable to verify the age of the person and provide one or more attestations regarding such to confirm that the person may legally be provided the medical service. Various configurations are possible and contemplated without departing from the scope of the present disclosure.

Moreover, in various implementations, the system 300 may control access using the identity information. For example, the system 300 may lock/unlock one or more rooms using the identity information, control access to one or more medications and/or information, and so on. Various configurations are possible and contemplated without departing from the scope of the present disclosure.

Although the system 300 is illustrated and described as including the insurance system electronic device 305, the payment system electronic device 306, and the medical records electronic device 307, it is understood that this is an example. In various implementations, one or more of these devices may be included without utilizing all. Various configurations are possible and contemplated without departing from the scope of the present disclosure.

Further, although the system 300 is described above as facilitating insurance and/or other payment processing and/or access to medical records contemporaneous with checking a person in for a medical service, it is understood that this is an example. In various implementations, the system 300 may perform these operations at different times (such as checking in a person upon arrival for a medical service, facilitating access to medical records during the medical service, verifying a person's identity after check in but before performance of a medical procedure to ensure that the medical procedure is performed on the same person who checked in, processing payment after the medical service, and so on). In some examples, the digital representation of the biometric and/or other digital representations of biometrics may be obtained for each operation, obtained once for an entire chain of operations, and so on. Various configurations are possible and contemplated without departing from the scope of the present disclosure.

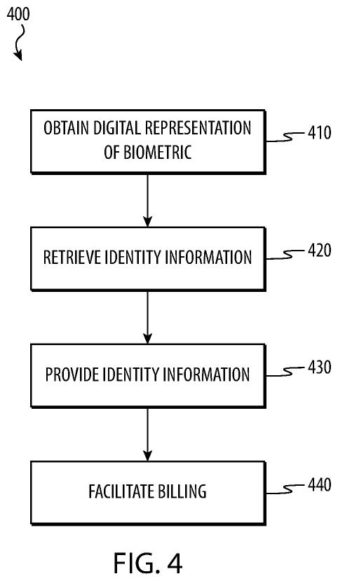

FIG. 4 is a flow chart illustrating a second example method 400 for biometric secured medical check in. This method 400 may be performed by the systems 100, 300 of FIGS. 1 and/or 3.

At operation 410, an electronic device (such as the identity system electronic device 102, 302 of FIGS. 1 and/or 3) may obtain a digital representation of a biometric. For example, the electronic device may receive the digital representation of the biometric from a biometric reader device, from another electronic device (such as the check in electronic device 101, 301 of FIGS. 1 and/or 3), and so on.

At operation 420, the electronic device may retrieve identity information using the digital representation of the biometric. The electronic device may retrieve the identity information from a storage component of the electronic device, from an external database, and so on.

At operation 430, the electronic device may provide the identity information. For example, the electronic device may provide the identity information to an electronic device operated by a medical service provider (such as the medical service electronic device 103, 303 of FIGS. 1 and/or 3), to an electronic device associated with a person (such as the check in electronic device 101, 301 of FIGS. 1 and/or 3) for display, editing, and/or authorization to pass on the identity information to another electronic device, and so on. The electronic device may check in a person for a medical service and/or facilitate such as part of providing the identity information.

At operation 440, the electronic device may facilitate billing. For example, the identity information may include and/or be associated with payment information (such as insurance information, one or more financial account numbers, and so on). The electronic device may retrieve the payment information and use the payment information to process payment (such as processing an insurance or financial account number payment for a medical service), provide payment information to another device to schedule billing using the payment information, and so on.

In various examples, this example method 400 may be implemented as a group of interrelated software modules or components that perform various functions discussed herein. These software modules or components may be executed within a cloud network and/or by one or more computing devices, such as the identity system electronic devices 102, 302 and/or the check in electronic devices 101, 301 of FIGS. 1 and/or 3.

Although the example method 400 is illustrated and described as including particular operations performed in a particular order, it is understood that this is an example. In various implementations, various orders of the same, similar, and/or different operations may be performed without departing from the scope of the present disclosure.

For example, operation 440 is illustrated and described as facilitating billing. However, it is understood that this is an example. In some implementations, facilitating billing may be replaced and/or supplemented with processing one or more payments (such as where insurance is billed but a payment for a copay is processed). Various configurations are possible and contemplated without departing from the scope of the present disclosure.

In some implementations, a system for biometric secured medical check in may include at least one non-transitory storage medium that stores instructions and at least one processor. The at least one processor may execute the instructions to receive a digital representation of a biometric of a person, use the digital representation of the biometric to retrieve identity information for the person, provide the identity information to a medical service electronic device to check the person in for a medical service, and process payment for the medical service using payment information stored in association with the identity information.

In various examples, the payment information may include insurance information for the person. In some such examples, the at least one processor may process the payment by submitting an insurance payment request using the insurance information. In a number of such examples, the at least one processor may process the payment by providing the insurance information to the medical service electronic device. In various such examples, the at least one processor may determine a copay associated with the medical service and the insurance information and obtain a payment from the person for the copay.

In a number of examples, the payment information may include a financial account number. In some such examples, the at least one processor may process the payment by charging the financial account number. In various such examples, the at least one processor may process the payment by providing the financial account number to the medical service electronic device.

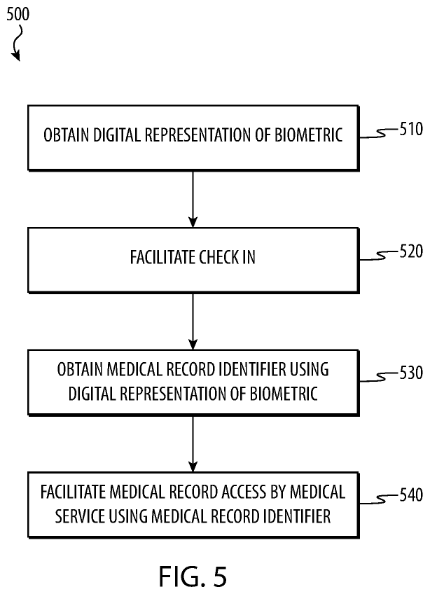

FIG. 5 is a flow chart illustrating a third example method 500 for biometric secured medical check in. This method 500 may be performed by the systems 100, 300 of FIGS. 1 and/or 3.

At operation 510, an electronic device (such as the identity system electronic devices 102, 302 of FIGS. 1 and/or 3) may obtain a digital representation of a biometric. At operation 520, the electronic device may use the digital representation of the biometric to facilitate check in for a medical service.

For example, the electronic device may use the digital representation of the biometric to retrieve associated identity information. The electronic device may provide the identity information to an electronic device operated by a medical service provider (such as the medical service electronic devices 103, 303 of FIGS. 1 and/or 3), which may use the identity information to check in a person for a medical service.

At operation 530, the electronic device may obtain a medical record identifier using the digital representation of the biometric. In some implementations, the medical record identifier may be included in and/or associated with the identity information.

At operation 540, the electronic device may facilitate medical record access by a medical service using a medical record identifier. For example, the electronic device may provide the medical record identifier and/or a specification of medical records requested to a medical records database (such as one that may be maintained by the medical records electronic device 307 of FIG. 3), receive and then provide one or more medical records to another electronic device (such as to the medical service electronic devices 103, 303 of FIGS. 1 and/or 3) and/or direct where such medical records should be received, and so on. By way of another example, the electronic device may provide the medical record identifier to another device (such as the medical service electronic devices 103, 303 of FIGS. 1 and/or 3) that may then communicate with the medical records database (such as one that may be maintained by the medical records electronic device 307 of FIG. 3) directly.

In various examples, this example method 500 may be implemented as a group of interrelated software modules or components that perform various functions discussed herein. These software modules or components may be executed within a cloud network and/or by one or more computing devices, such as the identity system electronic devices 102, 302 and/or the check in electronic device 101, 301 of FIGS. 1 and/or 3.

Although the example method 500 is illustrated and described as including particular operations performed in a particular order, it is understood that this is an example. In various implementations, various orders of the same, similar, and/or different operations may be performed without departing from the scope of the present disclosure.

For example, the method 500 is illustrated and described as facilitating check in and medical records access in separate operations. However, in some implementations, facilitation of medical records access and check in may be part of a single operation. Various configurations are possible and contemplated without departing from the scope of the present disclosure.

In various implementations, a system for biometric secured medical check in may include at least one non-transitory storage medium that stores instructions and at least one processor. The at least one processor may execute the instructions to receive a digital representation of a biometric of a person, use the digital representation of the biometric to retrieve identity information for the person, provide the identity information to a medical service electronic device to check the person in for a medical service, use the digital representation of the biometric to retrieve a medical record identifier for the person, and use the medical record identifier to facilitate access by the medical service electronic device to a medical record for the person stored by a medical records electronic device.

In some examples, the at least one processor may facilitate the access by providing the medical record identifier to the medical service electronic device. In other examples, the at least one processor may facilitate the access by providing the medical record identifier to the medical records electronic device and providing a response from the medical records electronic device to the medical service electronic device.

In a number of examples, the medical record may include a vaccination list. In some examples, the medical record may include at least part of a medical history. In various examples, the medical record may include an allergy list. In a number of examples, the medical record may include a current medication list. In some examples, the medical record may include a preferred pharmacy. In various examples, the medical record may include a list of the person's current medical service providers. In some implementations, the list may include contact information for the person's current medical service providers so that one or more of the person's current medical service providers may be notified regarding medical services provided to the person. In a number of examples, the medical record may include a list of the person's past medical service providers. In some examples, the medical record may include a lab diagnostic result, a lab imaging result, an order for lab tests, and so on. Various configurations are possible and contemplated without departing from the scope of the present disclosure.

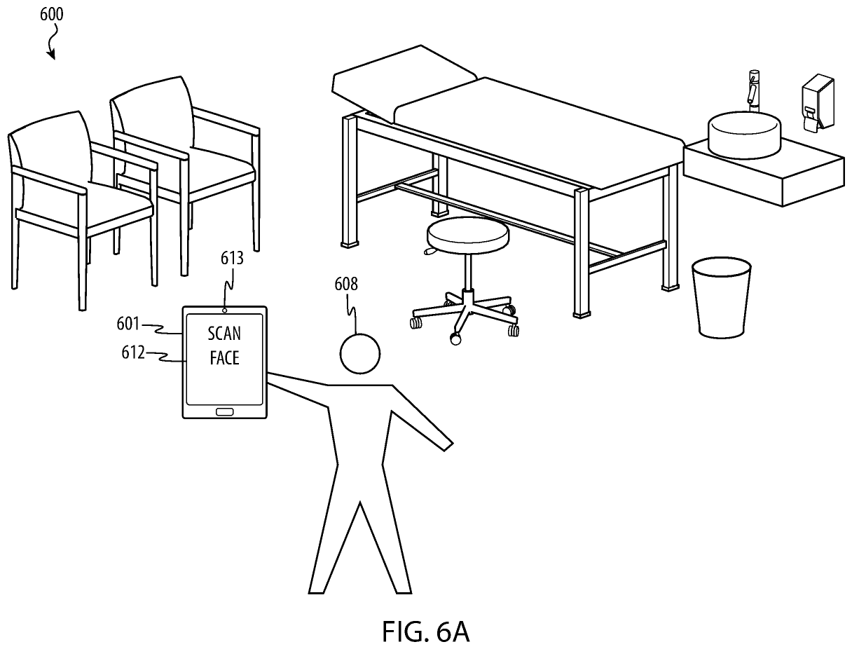

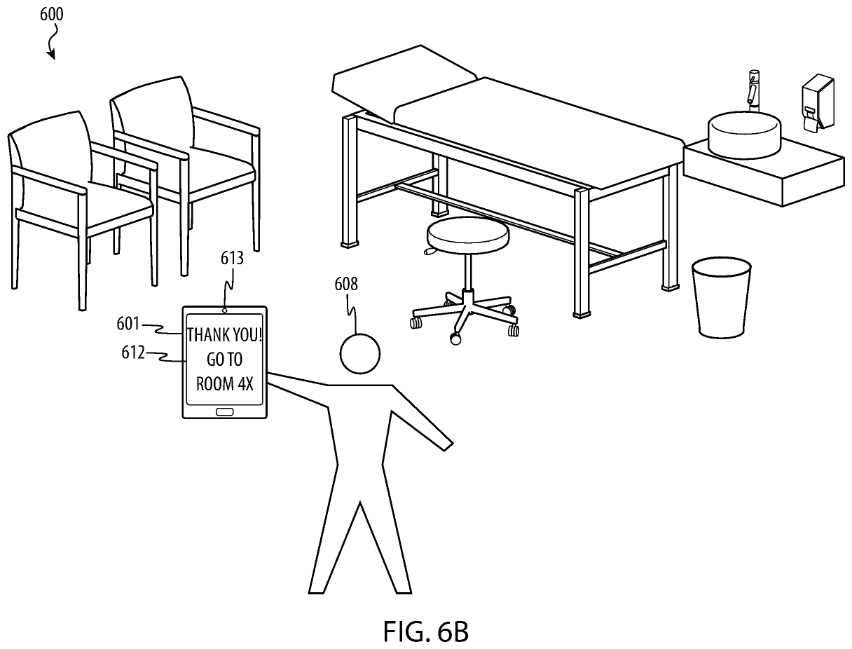

FIG. 6A depicts a third example system 600 for biometric secured medical check in. In this example, a person 608 may use a tablet computing device 601 in a doctor's waiting room. The tablet computing device 601 may include a 2D and/or 3D camera 613 and a screen 612. The tablet computing device 601 may execute an app and/or application that displays a message on the screen 612 prompting the person to scan an image of his face using the camera 613.

The person 608 may use the camera 613 to scan an image of his face. A digital representation of the image may be transmitted to an identity system. The identity system may receive the digital representation of the image, retrieve identity information using the digital representation of the image, and check in the person 608 for an appointment at the doctor's office (and/or facilitate such) by transmitting the identity information to a system at the doctor's office.

An acknowledgement of the check in may be transmitted to the tablet computing device 601, such as by the identity system, the system at the doctor's office, and so on. The acknowledgment may include instructions regarding the medical service. For example, as shown in FIG. 6B, the tablet computing device 601 may receive an acknowledgment and display such on the screen 612, indicating that the person 608 is to proceed to room 4X for the medical service.

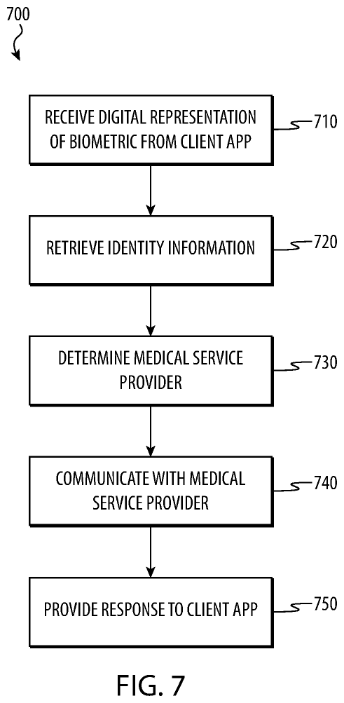

FIG. 7 is a flow chart illustrating a third example method 700 for biometric secured medical check in. This method 700 may be performed by the systems 100, 300, 600 of FIGS. 1, 3, and/or 6A and 6B.

At operation 710, an electronic device (such as the identity system electronic devices 102, 302 of FIGS. 1 and/or 3) may receive a digital representation of a biometric from a client app and/or application, such as a client app and/or application executing on the tablet computing device 601 of FIGS. 6A and 6B. At operation 720, the electronic device may use the digital representation of the biometric to retrieve identity information. At operation 730, the electronic device may determine a medical service provider.

For example, the electronic device may determine a medical service provider according to an input received from a person via the client app and/or application. By way of another example, the electronic device may determine a medical service provider using a location component of an electronic device on which the client app and/or application is executing and comparing that location to medical service provider locations. In yet another example, the electronic device may determine a medical service provider according to a network via which the digital representation of the biometric was received and determining a medical service provider location associated with that network. In still another example, the electronic device may determine a medical service provider according to network identifiers included in a transmission associated with receipt of the digital representation of the biometric and determining a medical service provider location indicated by the network identifiers. In additional examples, the electronic device may determine a medical service provider based on data included in the identity information.

At operation 740, the electronic device may communicate with the determined medical service provider. For example, the electronic device may check in a person for a medical service and/or facilitate such by providing the identity information and/or a portion thereof to a system of the medical service provider.

The electronic device may receive one or more responses from the system, such as a confirmation of check in, an acknowledgement of the check in including instructions regarding the medical service, and so on. At operation 750, the electronic device may provide the response to the client app and/or application.

In various examples, this example method 700 may be implemented as a group of interrelated software modules or components that perform various functions discussed herein. These software modules or components may be executed within a cloud network and/or by one or more computing devices, such as the identity system electronic devices 102, 302 of FIGS. 1 and/or 3, the check in electronic devices 101, 301 of FIGS. 1 and/or 3, and/or the tablet computing device 601 of FIGS. 6A and 6B.

Although the example method 700 is illustrated and described as including particular operations performed in a particular order, it is understood that this is an example. In various implementations, various orders of the same, similar, and/or different operations may be performed without departing from the scope of the present disclosure.

For example, the method 700 is illustrated and described as providing a response to the client app and/or application. However, it is understood that this is an example. In some implementations, the response may be provided to another electronic device indicated in the identity information. In other implementations, no response may be provided. Various configurations are possible and contemplated without departing from the scope of the present disclosure.

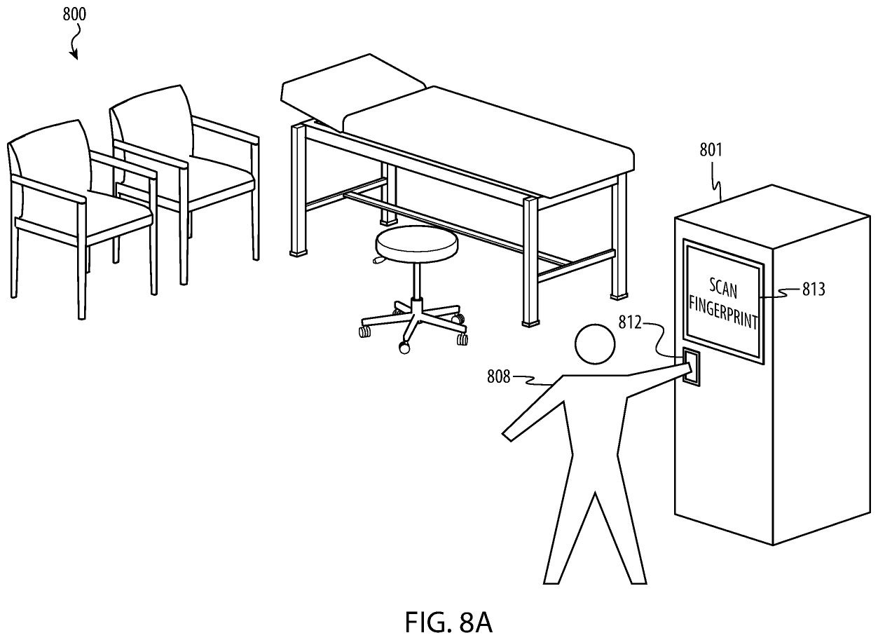

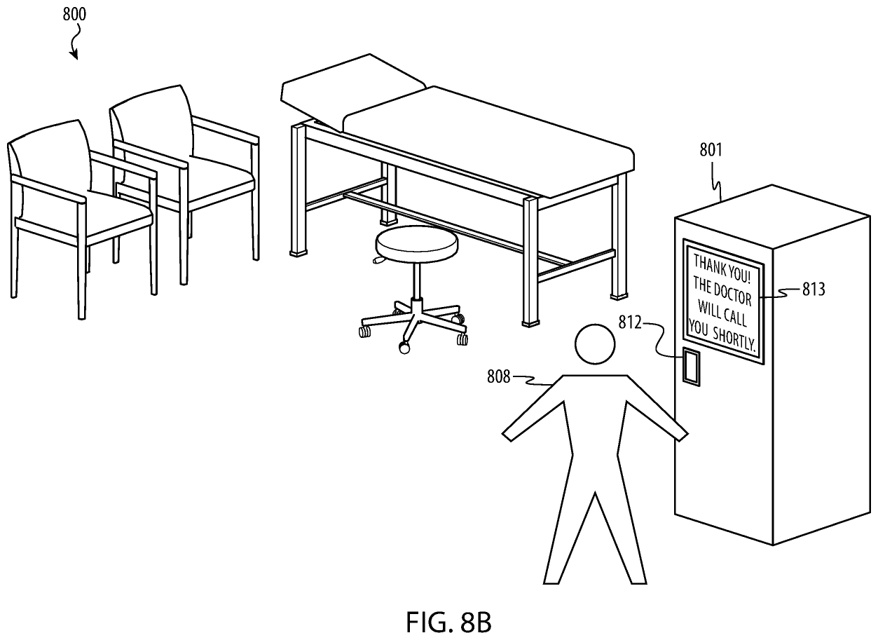

FIG. 8A depicts a fourth example system 800 for biometric secured medical check in. In this example, a person 808 may use a kiosk 801 or other station provided in a doctor's waiting room to check in for a medical service appointment. The kiosk 801 may include a fingerprint scanner 812 and a screen 813.

The kiosk 801 may execute an app and/or application that displays a message on the screen 813 prompting the person to scan his fingerprint using the fingerprint scanner 812. The person 808 may use the fingerprint scanner 812 to scan his fingerprint. A digital representation of the scan may be transmitted to an identity system. The identity system may receive the digital representation of the image, retrieve identity information using the digital representation of the image, and check in the person 808 for an appointment at the doctor's office (and/or facilitate such) by transmitting the identity information to a system at the doctor's office. An acknowledgement of the check in may be transmitted to the kiosk 801, such as by the identity system, the system at the doctor's office, and so on. For example, as shown in FIG. 8B, the kiosk 801 may receive an acknowledgment and display such on the screen 813, indicating that the doctor will call the person for the medical service shortly.

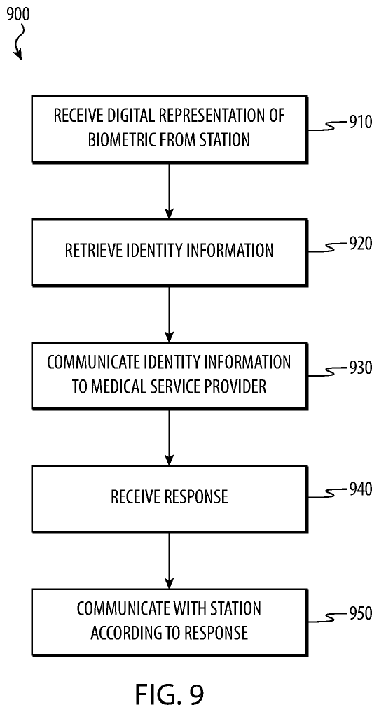

FIG. 9 is a flow chart illustrating a fourth example method 900 for biometric secured medical check in. This method 900 may be performed by the systems 100, 300, 800 of FIGS. 1, 3, and/or 8A and 8B.

At operation 910, an electronic device (such as the identity system electronic devices 102, 302 of FIGS. 1 and/or 3) may receive a digital representation of a biometric from a station, such as the kiosk 801 of FIGS. 8A and 8B. At operation 920, the electronic device may use the digital representation of the biometric to retrieve identity information. At operation 930, the electronic device may communicate the identity information to a system of a medical service provider, such as the doctor's office at which the kiosk 801 of FIGS. 8A and 8B is located.

At operation 940, the electronic device may receive one or more responses from the system, such as a confirmation of a medical service check in, an acknowledgement of a medical service check in including instructions regarding the medical service, and so on. At operation 950, the electronic device may communicate with the station according to the response.

For example, the response may indicate to instruct the station to display information. As such, the electronic device may communicate with the station to display information. In other examples, the response may indicate to obtain additional information (such as one or more selections, payments, authorizations, and so on). As such, the electronic device may communicate with the station to obtain the additional information. Various configurations are possible and contemplated without departing from the scope of the present disclosure.

In various examples, this example method 900 may be implemented as a group of interrelated software modules or components that perform various functions discussed herein. These software modules or components may be executed within a cloud network and/or by one or more computing devices, such as the identity system electronic devices 102, 302 of FIGS. 1 and/or 3, the check in electronic devices 101, 301 of FIGS. 1 and/or 3, and/or the kiosk 801 of FIGS. 8A and 8B.

Although the example method 900 is illustrated and described as including particular operations performed in a particular order, it is understood that this is an example. In various implementations, various orders of the same, similar, and/or different operations may be performed without departing from the scope of the present disclosure.

For example, the method 900 is illustrated and described as having the electronic device communicate with the station according to the response. However, it is understood that this is an example. In some implementations, the electronic device may not communicate with the station according to the response. In various examples, the electronic device may instead communicate with another electronic device, such as an electronic device indicated in the identity information and/or otherwise associated with the identity information. Various configurations are possible and contemplated without departing from the scope of the present disclosure.

Although medical services are described numerous times above in the context of an appointment at a doctor's office, it is understood that this is an example. The present disclosure may be used in contexts other than medical services without departing from the scope of the present disclosure and medical services may be any kind of service provided in relation to medicine with or without a scheduled appointment. In some implementations, arriving at a pharmacy and/or other automated, partially automated, and/or non-automated medicine and/or medical product dispensary may constitute a check in for a medical service without departing from the scope of the present disclosure.

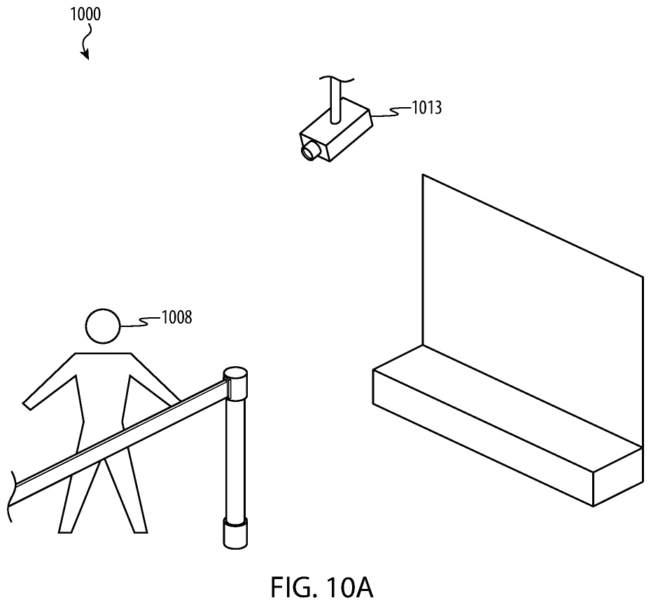

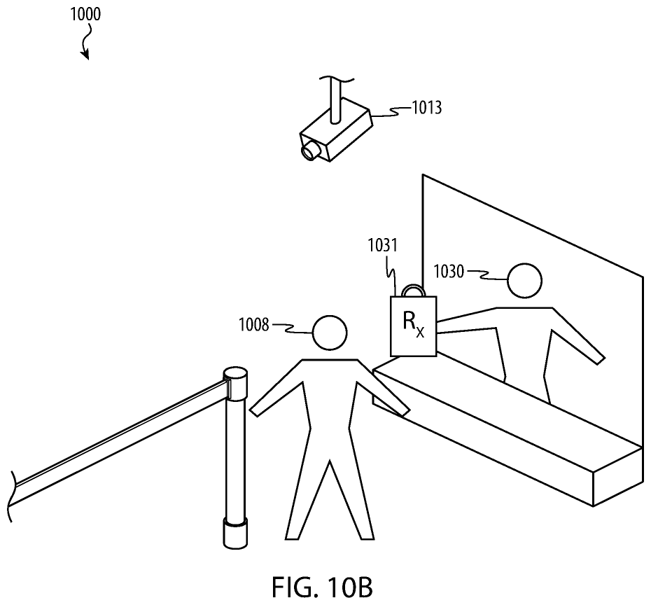

For example, FIG. 10A depicts a fifth example system 1000 for biometric secured medical check in. In this example, a person 1008 may wait in a line to approach a pharmacy counter. A camera 1013 may capture a digital representation of a face of the person 1008. Identity information for the person may be retrieved using the digital representation of the person's face. As shown in FIG. 10B, the identity information may be used to determine one or more medications 1031 to provide to the person 1008 and a pharmacist 1030 and/or other delivery mechanism may be directed to provide such.

For example, the identity information may be used to access medications 1031 that the person 1008 has requested. By way of another example, the identity information may be used to retrieve a medical record identifier for the person and access prescriptions indicated in a medical record accessible from a medical records system using the medical record identifier. In such an example, the medications 1031 may be selected using such prescriptions (such as a prescription that has been called into the pharmacy for the person 1008, a refill that the person 1008 has available and is due to pick up according to when a previous prescription fill would have been finished, and so on). In some examples, the identity information may be used to verify that the person is allowed to obtain the medication 1031, such as verifying an age of the person 1008 for prescriptions that may only legally be provided to people of a certain age (such as 18 years of age, 21 years of age, and so on), verifying that the person has not already obtained more than a regulated amount of a medical product (such as prescription cold medicines that may be restricted to a certain amount obtained in a single day, week, and so on), verifying that a prescription does not have an adverse interaction with another medication the person is indicated as taking in a medical record and/or the identity information, and so on.

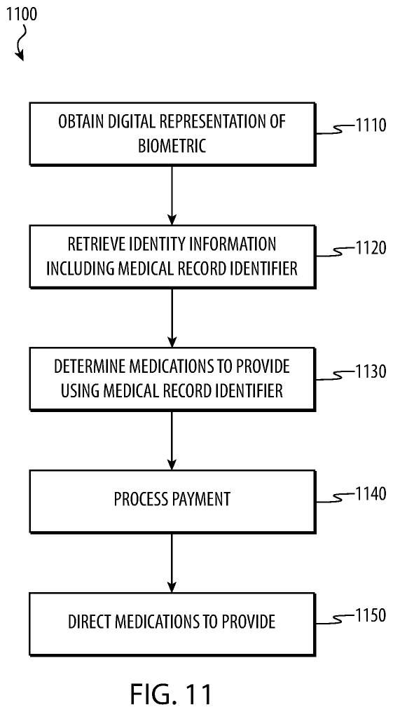

FIG. 11 is a flow chart illustrating a fifth example method 1100 for biometric secured medical check in. This method 1100 may be performed by the systems 100, 300, 1000 of FIGS. 1, 3, and/or 10A and 10B.

At operation 1110, an electronic device (such as the identity system electronic devices 102, 302 of FIGS. 1 and/or 3) located at a pharmacy and/or other medicine and/or medical product dispensing location may obtain a digital representation of a biometric for a person. At operation 1120, the electronic device may use the digital representation of the biometric to retrieve identity information. The identity information may include and/or be associated with a medical record identifier.

At operation 1130, the electronic device may determine one or more medications to provide using a medical record identifier. At operation 1140, the electronic device may process payment for the medications (such as using one or more credit card and/or other payment terminals, using payment information included in and/or associated with the identity information, and so on). At 1150, the electronic device may direct the medications to provide. For example, the electronic device may transmit and/or otherwise present a message to a pharmacist or other person regarding the medications to provide and/or the person to whom to provide the medications.

In various examples, this example method 1100 may be implemented as a group of interrelated software modules or components that perform various functions discussed herein. These software modules or components may be executed within a cloud network and/or by one or more computing devices, such as the identity system electronic devices 102, 302 of FIGS. 1 and/or 3, the check in electronic devices 101, 301 of FIGS. 1 and/or 3, and/or one or more electronic devices of the system 1000 of FIGS. 10A and 10B.

Although the example method 1100 is illustrated and described as including particular operations performed in a particular order, it is understood that this is an example. In various implementations, various orders of the same, similar, and/or different operations may be performed without departing from the scope of the present disclosure.

For example, the method 1100 is illustrated and described as processing payment. However, in some examples, the method 1100 may instead facilitate payment processing, omit payment processing, and/or perform other actions. Various configurations are possible and contemplated without departing from the scope of the present disclosure.

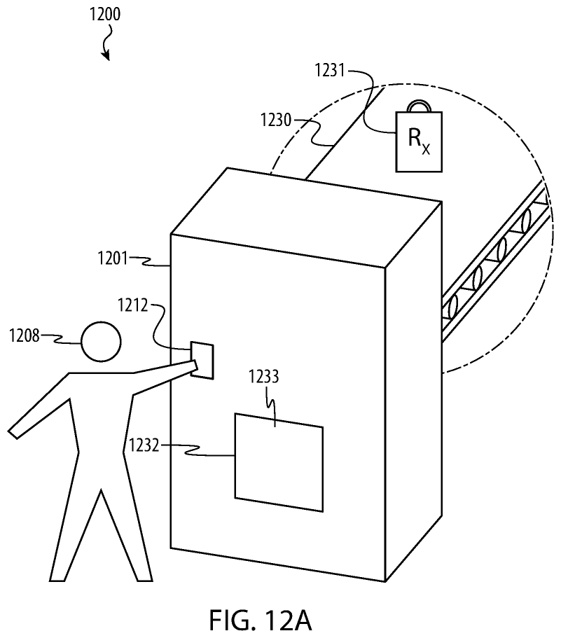

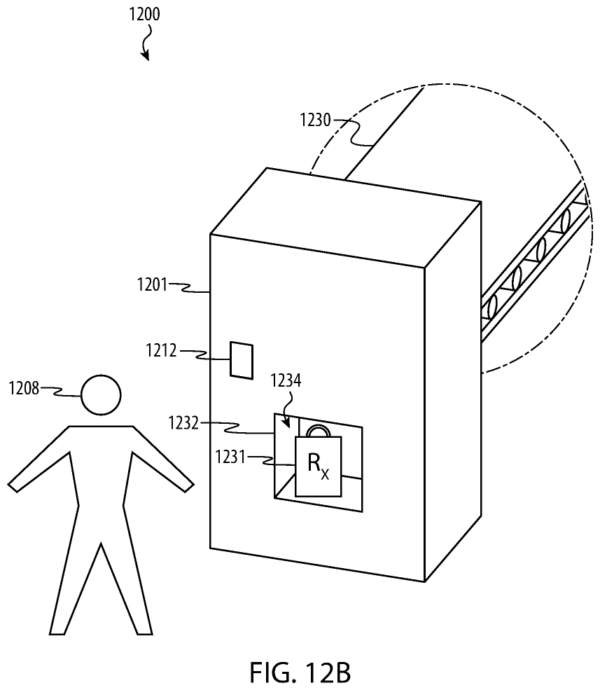

FIG. 12A depicts a sixth example system 1200 for biometric secured medical check in. In this example, a person 1208 may use a medical product automated dispensing device 1201 (such as a vending machine) to obtain one or more medical products 1231. The person 1208 may specify requested medical products and/or medical products 1231 may be determined for the person 1208.

The medical product automated dispensing device 1201 may include a fingerprint pad 1212. The person 1208 may use the fingerprint pad 1212 to provide one or more fingerprint images. The medical product automated dispensing device 1201 may obtain a digital representation of the fingerprint image, use such to retrieve identity information for the person, determine one or more medical products to dispense, obtain such medical products using one or more transport mechanisms, and provide the medical products.

For example, in this example, the medical product automated dispensing device 1201 may include a hatch 1232 that is covered by a door 1233. The medical product automated dispensing device 1201 may be connected to a medical product storage area via a conveyor belt 1230 and/or other delivery system hidden from the person 1208 on the other side of a wall. The medical product automated dispensing device 1201 may be configured to receive the medical products 1231 into the area of the hatch 1232 blocked by the door 1233 via the conveyor belt 1230. As shown in FIG. 12B, the medical product automated dispensing device 1201 may then withdraw the door 1233 to expose an aperture 1234 of the hatch 1232 where the medical products 1231 are located so that the person 1208 has access to the medical products 1231.

Although the above describes the medical product automated dispensing device 1201 as determining one or more medical products to dispense, it is understood that this is an example. In some implementations, the medical product automated dispensing device 1201 may provide the person 1208 a list of such medical products and allow the person 1208 to select among the list. The medical product automated dispensing device 1201 may then obtain and provide the selected metical products. Various configurations are possible and contemplated without departing from the scope of the present disclosure.

Further, although the above illustrates and describes a conveyor belt 1230, it is understood that this is an example and that other delivery systems may be used without departing from the scope of the present disclosure. For example, one or more rotating coils may be used to move one or more medical products from a shelf such that the medical product falls into an aperture that is accessible by the person 1208. Various configurations are possible and contemplated without departing from the scope of the present disclosure.

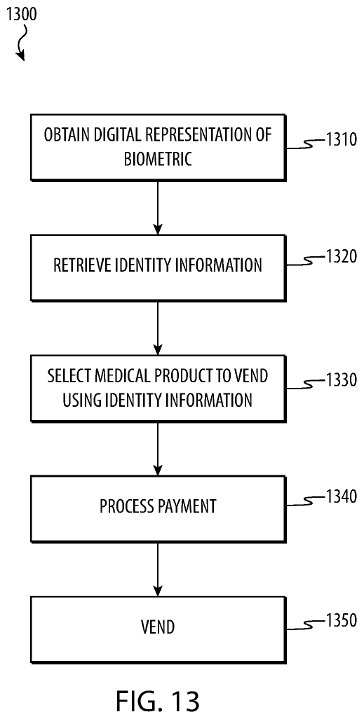

FIG. 13 is a flow chart illustrating a fifth example method 1300 for biometric secured medical check in. This method 1300 may be performed by the systems 100, 300, 1200 of FIGS. 1, 3, and/or 12A and 12B.

At operation 1310, an electronic device (such as the identity system electronic devices 102, 302 of FIGS. 1 and/or 3 and/or the medical product automated dispensing device 1201 of FIG. 12) may obtain a digital representation of a biometric. At operation 1320, the electronic device may use the digital representation of the biometric to retrieve associated identity information. At operation 1330, the electronic device may use the identity information to select one or more medical products to vend.

At operation 1340, the electronic device may process one or more payments for the medical product. For example, the electronic device may include a credit/debit card reader, a bill collector, and so on and may use such to process payment. By way of another example, the electronic device may use payment information associated with the identity information to process payment. At operation 1350, the electronic device may vend the medical product.

In various examples, this example method 1300 may be implemented as a group of interrelated software modules or components that perform various functions discussed herein. These software modules or components may be executed within a cloud network and/or by one or more computing devices, such as the identity system electronic devices 102, 302 of FIGS. 1 and/or 3, the check in electronic devices 101, 301 of FIGS. 1 and/or 3, and/or one or more electronic devices of the system 1200 of FIGS. 12A and 12B.

Although the example method 1300 is illustrated and described as including particular operations performed in a particular order, it is understood that this is an example. In various implementations, various orders of the same, similar, and/or different operations may be performed without departing from the scope of the present disclosure.

Although the method 1300 is illustrated and described in the context of a medical product vending machine, it is understood that this is an example. In various implementations, a variety of automated and/or semi-automated medical product providing systems other than vending machines may perform the method 1300. Various configurations are possible and contemplated without departing from the scope of the present disclosure.

Although the above is illustrated and described within the context of biometric secured medical check in, it is understood that this is an example. In various implementations, the systems 100, 300, 1200 of FIGS. 1, 3, and/or 12A and 12B and/or other techniques discussed herein may be used in a variety of contexts without departing from the scope of the present disclosure.

For example, identity systems (identity system electronic devices 102, 302 of FIGS. 1 and/or 3) may be used to control access to identity information (such as using digital images of one or more fingerprints, irises, faces, and/or other biometrics to identify people, authenticate identity, access related identity information, and so on) in order to facilitate a variety of functions. The identity system may interact with one or more electronic devices in order to determine and/or otherwise verify and/or authenticate a person's identity, validate a driver's license or other identity token for a person or provide information therefrom, validate an insurance card for a person or provide information thereof or function as a replacement for such, process payments using one or more credit cards and/or other financial accounts and/or provide information therefrom, validate one or more credit cards and/or other financial accounts and/or verify authorization to use such, verify boarding pass and/or other ticketing information (such as plane, bus, or train tickets; tickets to enter sporting or other venues; and so on), enable picking up of a rental vehicle, process payment for goods or services such as food and drinks, determine access to buildings, rooms, and/or other locations, and so on.

In various implementations, the identity system may interact with one or more electronic devices in order to perform various actions for patients whose identity information is accessible to the identity system. For example, the identity system may perform a variety of identification functions, such as positively identifying patients with confidence at one or more stages of their healthcare journey. By way of another example, the identity system may perform a variety of security functions, such as improving security and reducing fraud while minimizing and/or otherwise reducing cumbersome security protocols. In yet another example, the identity system may perform a variety of patient experience functions, such as enabling a seamless visit that focuses patients and staff on care rather than paperwork.

The identity system may connect the patient journey with an obtained digital representation of a biometric, such as a digital representation of a glance. The identity system may streamline the patient experience across the healthcare ecosystem and beyond with a unified biometric patient identifier, visits using biometrics or biometrics along with another identifier (such as a password, a physical item such as a card, and so on), and/or secure payments. The identity system may enable patients to check into an emergency room and/or other medical service provider location (such as by validating identity, providing access to medical records and/or insurance, and so on), visit one or more labs for testing (which may ensure accuracy, reduce duplicate testing, and so on), be discharged to a specialist (which may involve enabling the patient to pay for a visit, receive instructions, and so on), receive services on arrival for an appointment (such as enabling self-service, paperless check in, verification, payment, and so on), pick up one or more prescriptions (in some examples allowing a prescription to be automatically and/or semi-automatically dispensed in response to a received digital representation of a biometric), share visit information with authorized physicians and/or other medical service providers, and so on.

In some implementations, the identity system may be used to reimagine the pharmacy experience. People may be able to safely access medication anywhere at any time. The identity system may streamline the person's experience by increasing access to controlled and/or prescription medication in the store and/or beyond.

The identity system may enable innovation for the in-store medical product experience. The identity system may enable 24/7 medication pickup, which may reduce staffing costs and/or improve patient convenience at clinics and/or pharmacies. Integrated storage lockers and/or other devices may enable remote ordering (such as online, by phone, by text message, and so on) and pickup using digital representations of biometrics. The identity system may enable secure access to controlled substances and/or sensitive areas, simplify staff workflows, mitigate risk with better access, and oversight, and so on.

The identity system may enable pharmacies and/or other medical product providers to grow their retail footprint. For example, automated dispensing apparatuses (such as vending machines and so on) may be used to dispense behind-the-counter products and over-the-counter products at airports, stadiums, and/or other locations. The identity system may provide new ways to reach customers, such as via pharmacy delivery, dispensing solutions, and so on. The identity system may also enable embedded loyalty programs, which may drive behavioral change by incentivizing patients to stay healthy and adhere to health programs.

In various implementations, the identity system may enable reimagining of medical service provider employee experiences. The identity system may enable medical service provider employees a less burdensome and more secure way to go about their workday. This may boost employee satisfaction and/or data security across the healthcare ecosystem with a unified biometric identifier, access to rooms and/or other locations using biometrics or biometrics along with another identifier (such as a password, a physical item such as a card, and so on), workstations, substances, and so on. For example, the identity system may enable access of authorized personnel to a room, locker, or other storage area using biometrics or biometrics along with another identifier (such as a password, a physical item such as a card, and so on) where items such as prescription and/or other medications may be stored and may log who obtains access and/or any items accessed and/or removed. The identity system may integrate existing medical service provider systems to reduce redundant tasks, such as by the identity system integrating information between a scheduling system and a billing system so that staff does not need to obtain patient information from the patient (and/or the scheduling system) that is already in the scheduling system in order to enter the information into the billing system.

For example, the identity system may enable an employee to walk into a clinic or other medical location without an identification card, access specialty areas (which may improve physical security without adding additional hassle), log into computers and/or other equipment (such may enable employees to spend less time accessing critical and/or other data), pay for goods or services (such as coffee, cafeteria food, and so on) without providing cash or cards (which may enable employees to enjoy breaks without holdups), access medication carts or other areas (which may decentralize access of controlled substances), head to hospitals or other locations for patient visits (which may remove excess access checkpoints), visit a lab for patient results (which may control contamination risks, patient results visibility, and so on), and so on. Various configurations are possible and contemplated without departing from the scope of the present disclosure.

As discussed above, the present disclosure also relates to biometric identity system integration of medical service provider systems. This will now be elaborated in further detail.

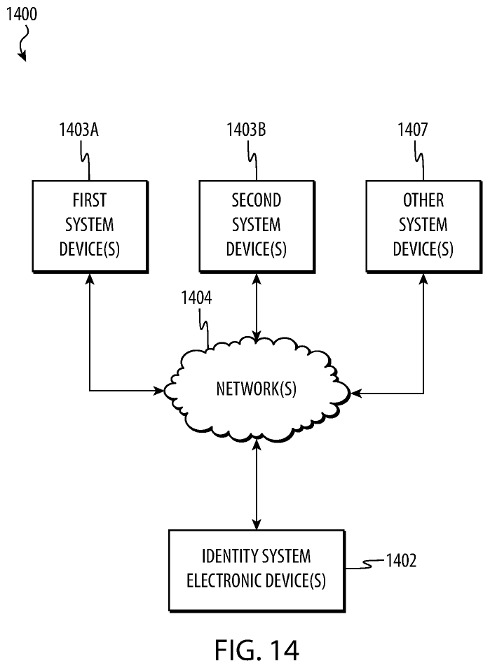

FIG. 14 depicts a first example system 1400 for biometric identity system integration of medical service provider systems. The system 1400 may include one or more first system devices 1403A and one or more second system devices 1403B that are operable to communicate with one or more identity system electronic devices 1402 via one or more wired and/or wireless communication networks 1404.

The first system device 1403A and the second system device 1403B may be configured to use the identity system electronic device 1402 for biometric identification. For example, each of the first system device 1403A and the second system device 1403B may be configured to provide captured biometric data to the identity system electronic device 1402 that may identify a person using the biometric data and provide one or more attestations (such as an identity of the person, identity information associated with the identity stored by the identity system and/or another system for which the identity system controls and/or directs data access, confirmations regarding identity information like a verification that a person is of a minimum age or has insurance coverage, and so on). Various configurations are possible and contemplated without departing from the scope of the present disclosure.

The identity system electronic device 1402 may be operable to integrate the first system device 1403A and the second system device 1403B. The identity system electronic device 1402 may communicate with the first system device 1403A and the second system device 1403B to provide information stored by the identity system electronic device 1402 and/or by one or more other system devices 1407 for which identity system electronic device 1402 is able to control and/or direct data access. In this way, the first system device 1403A and the second system device 1403B may have the enhanced functions of access to and/or of exchange data that they would not otherwise be able to perform. This may improve operation of the first system device 1403A and the second system device 1403B as useful data may not be lacking in one or more of the systems, redundant and/or out of date data may be eliminated and/or reduced, redundant components may be eliminated that would otherwise be used for such redundant data to be entered and/or stored, and so on. The first system device 1403A and the second system device 1403B may be more efficient due to reduced or eliminated unnecessary data entry, reduced or eliminated lack of access to data and/or up to date data, and so on. Various configurations are possible and contemplated without departing from the scope of the present disclosure.

In various embodiments, the identity system electronic device 1402 may communicate with the first system device 1403A and the second system device 1403B in order to exchange and/or direct the exchange of information. The identity system electronic device 1402 may maintain biometric data associated with identities in order to verify the identities for the medical service provider systems, such as for the first system device 1403A and the second system device 1403B. In various examples, the identity system electronic device 1402 may receive a request for identity information from the first system device 1403A and arrange for the identity information to be provided to the first system device 1403A and the second system device 1403B upon determining that the request is authorized. In some examples, the identity system electronic device 1402 may receive an update to the identity information from the first system device 1403A and arrange for the update to be provided to the second system device 1403B. In a number of examples, the identity system electronic device 1402 may determine that the first system device 1403A stores identity information that is missing from the second system device 1403B and may arrange for the identity information to be provided to the second system device 1403B. Various configurations are possible and contemplated without departing from the scope of the present disclosure.

The identity system electronic device 1402 may be any kind of electronic device and/or cloud and/or other computing arrangement and may include one more processing units, communication units, non-transitory storage media, and/or other components. The processing unit may execute one or more sets of instructions stored in the non-transitory storage media to perform various functions, such as storing biometric data for people and associated identity information (such as one or more names, addresses, telephone numbers, social security numbers, patient identification numbers or other identifiers, insurance data, financial data, medical histories, and so on), receiving one or more digital representations of biometrics, matching one or more received digital representations of biometrics to stored biometric data, retrieving identity information associated with stored biometric data matching one or more received digital representations of biometrics, providing retrieved identity information, communicating with the first system device 1403A and/or the second system device 1403B (and/or the other system device 1407) via the network 1404, and so on. Similarly, the first system device 1403A and/or the second system device 1403B may be any kind of electronic device and/or cloud and/or other computing arrangement and may include one more processing units, communication units, non-transitory storage media, biometric reader devices and/or other components.

Although the system 1400 is described in the context of integrating medical service provider systems, it is understood that this is an example. In other embodiments, the techniques of the present disclosure may be used to integrate other systems outside of the medical service provider context. Various configurations are possible and contemplated without departing from the scope of the present disclosure.

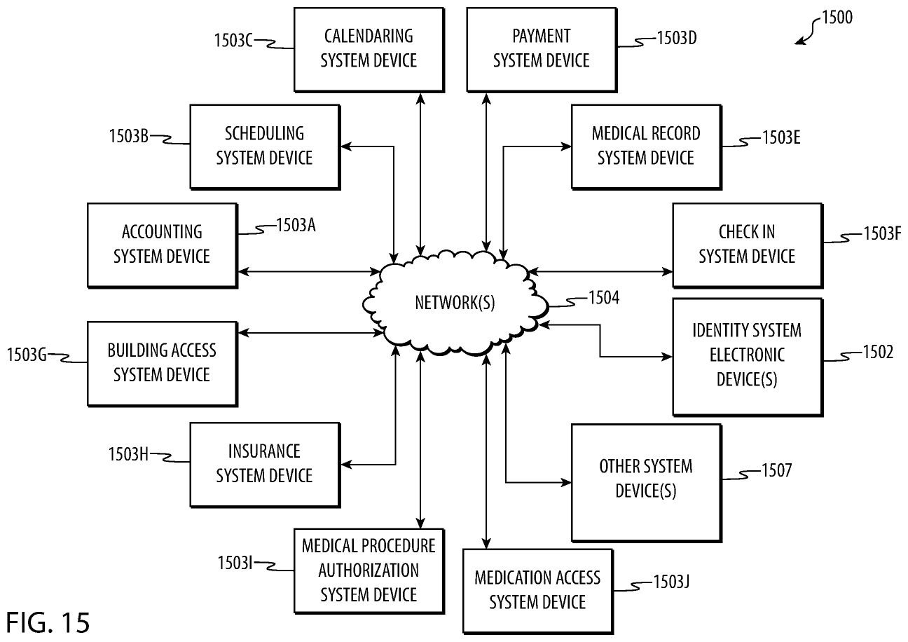

FIG. 15 depicts a second example system 1500 for biometric identity system integration of medical service provider systems. The system 1500 may include a number of different medical service provider systems that are operable to communicate with one or more identity system electronic devices 1502 via one or more wired and/or wireless communication networks 1504. For example, these different systems may include one or more accounting system devices 1503A, scheduling system devices 1503B, calendaring system devices 1503C, payment system devices 1503D, medical record system devices 1503E, check in system devices 1503F, building access system devices 1503G, insurance system devices 1503H, medical procedure authorization system devices 1503I, medication access system devices 1503J, and so on.

In various examples, the accounting system device 1503A may be operable to track money owed to a medical service provider for medical services performed and/or money owed by the medical service provider for various goods and/or services. In some examples, the scheduling system device 1503B may be operable to schedule medical services for patients and/or employees to provide those scheduled medical services. In a number of examples, the calendaring system device 1503C may be operable to manage calendars for one or more medical service provider employees. In various examples, the payment system device 1503D may be operable to process payment for money owed to the medical service provider. In some examples, the medical record system device 1503E may be operable to maintain patient medical records for the medical service provider. In a number of examples, the check in system device 1503F may be operable to check in patients for appointments with the medical service provider. In various examples, the building access system device 1503G may be operable to control access to one or more rooms and/or other gates and/or other access mechanisms in a facility for the medical service provider. In some examples, the insurance system device 1503H may be operable to code, submit, track, and/or otherwise process various insurance claims related to medical services provided by the medical service provider. In a number of examples, the medical procedure authorization system device 1503I may be operable to authorize medical procedures to be performed on various patients and/or by various employees. In various examples, the medication access system device 1503J may be operable to control and/or log access to various medications.

One or more of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J may be configured to use the identity system electronic device 1502 for biometric identification. For example, one or more of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J may be configured to provide captured biometric data to the identity system electronic device 1502 that may identify a person using the biometric data and provide one or more attestations (such as an identity of the person, identity information associated with the identity stored by the identity system electronic device 1502 and/or another system (such as one or more other system devices 1507, which may include one or more centralized medical records repositories) for which the identity system electronic device 1502 controls and/or directs data access (such as where the identity system electronic device 1502 directs the other system device 1507 to directly communicate data to a system instead of providing the data to the identity system electronic device 1502 to then pass on to the system), confirmations regarding identity information (like a verification that a person is of a minimum age or has insurance coverage, and so on). In some situations, the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J may use the identity system electronic device 1502 to verify the identity of a patient, obtain identity information and/or confirmations about identity information for the patient, and so on. In other situations, the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J may use the identity system electronic device 1502 to verify the identity of an employee (such as a doctor, nurse, support staff, and so on), obtain identity information and/or confirmations about identity information for the employee (such as an employee's access rights to a patient's medical records, an employee's medical certifications and/or qualifications, and so on), and so on.

As the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J may each communicate with the identity system electronic device 1502, the identity system electronic device 1502 may be used to integrate the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J. The identity system electronic device 1502 may communicate with the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J to provide information stored by the identity system electronic device 1502 and/or by other systems for which the identity system electronic device 1502 is able to control and/or direct data access. In this way, the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J may have the enhanced functions of access to and/or exchange of data that they would not otherwise be able to perform. This may improve operation of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J as useful data may not be lacking in one or more of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J, redundant and/or out of date data may be eliminated and/or reduced, redundant components may be eliminated that would otherwise be used for such redundant data to be entered and/or stored, and so on. The accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J may be more efficient due to reduced or eliminated unnecessary data entry, reduced or eliminated lack of access to data and/or up to date data, and so on.

The identity system electronic device 1502 may communicate with accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J in order to exchange and/or direct the exchange of information. The identity system electronic device 1502 may maintain biometric data associated with identities in order to verify the identities for the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J.

The identity system electronic device 1502 may exchange a variety of different information between the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J under a variety of different conditions and/or upon the occurrence of a variety of different triggering conditions. In some implementations, the identity system electronic device 1502 may exchange such information according to specifications included in identity information and/or stored by one or more of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J. Such specifications may include the information to exchange, permissions regarding information that may be exchanged, what people and/or systems with whom to share the information, conditions under which information is to be shared, how information is to be shared, and so on.

In various examples, the identity system electronic device 1502 may receive a request for identity information from a first system of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and the medication access system device 1503J. The identity system electronic device 1502 may arrange for the identity information to be provided to the first system and a second system of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and the medication access system device 1503J upon determining that the request is authorized.

In some implementations, the request may include a digital representation of a biometric and the identity system electronic device 1502 may determine that the request is authorized when the digital representation of the biometric matches biometric data associated with an identity corresponding to the identity information or with an identity that has permission to access the identity information. In a number of implementations, the identity system electronic device 1502 arranges to provide the identity information by transmitting the identity information to one or more of the first system and/or the second system itself. In various implementations, the identity system electronic device 1502 arranges to provide the identity information by instructing another electronic device to transmit the identity information to one or more of the first system and/or the second system. In a number of implementations, the identity system electronic device 1502 may determine whether or not the second system is authorized to receive the identity information before arranging for the identity information to be provided. Various configurations are possible and contemplated without departing from the scope of the present disclosure.

In some examples, the identity system electronic device 1502 may receive an update to the identity information from the first system of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and the medication access system device 1503J. The identity system electronic device 1502 may arrange for the update to be provided to the second system of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and the medication access system device 1503J.

The update may include one or more various kinds of additions to the identity information, changes to the identity information, and so on. The update may be a name, an address, a medical record, insurance information, payment information, and so on.

In some implementations, the update may be received from a person associated with an identity corresponding to the identity information. In various implementations, the identity information may correspond to a first identity and the update may be received from a person associated with a second identity that is authorized to modify the identity information.

In various implementations, the identity system electronic device 1502 may determine whether or not the update is authorized. For example, the identity system electronic device 1502 may determine whether or not the update is authorized by comparing a received digital representation of a biometric to a respective biometric associated with an identity corresponding to the identity information or an identity that is authorized to modify the identity information. The identity system electronic device 1502 may determine whether or not the update is authorized prior to arranging for the update to be provided to the second system. The identity system electronic device 1502 may update the identity information upon determining that the update is authorized. Various configurations are possible and contemplated without departing from the scope of the present disclosure.

In a number of examples, the identity system electronic device 1502 may determine that the first system of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and the medication access system device 1503J stores identity information that is missing from the second system of the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and the medication access system device 1503J. The identity system electronic device 1502 may arrange for the identity information to be provided to the second system.

In some implementations, the identity system electronic device 1502 may receive an identity verification request including a digital representation of a biometric and may provide a response based on a comparison of the digital representation of the biometric to at least a portion of biometric data maintained by the identity system electronic device 1502. The response may identify a respective identity that is associated with respective biometric data that matches the digital representation of the biometric.

In various implementations, the identity system electronic device 1502 may determine that the identity information is covered by a specification listing information to be duplicated in the first system and the second system. In a number of implementations, the identity system electronic device 1502 may determine that the second system is covered by a specification listing devices on which to duplicate information from the first system. In some implementations, the first system may not be configured to and/or capable of transmitting the identity information to the second system absent involvement of the identity system electronic device 1502. Various configurations are possible and contemplated without departing from the scope of the present disclosure.

The identity system electronic device 1502 may be any kind of electronic device and/or cloud and/or other computing arrangement and may include one more processing units, communication units, non-transitory storage media, and/or other components. The processing unit may execute one or more sets of instructions stored in the non-transitory storage media to perform various functions, such as storing biometric data for people and associated identity information (such as one or more names, addresses, telephone numbers, social security numbers, patient identification number or other identifier, insurance data, financial data, medical history, and so on); receiving one or more digital representations of biometrics; matching one or more received digital representations of biometrics to stored biometric data; retrieving identity information associated with stored biometric data matching one or more received digital representations of biometrics; providing retrieved identity information; communicating with the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J via the network 1504, and so on. For example, in some implementations, the identity system electronic device 1502 may be a remote device that communicates with one or more of the other devices of the system 1500 via the network 1504. In other implementations, the identity system electronic device 1502 may include a local device and a backend or similar device where the local interacts locally with one or more of the other devices of the system 1500 and the backend (such as where the local device is operable to perform biometric capture and/or other functions with a subset of data stored by the backend and interact with the backend to perform other functions). In some implementations, the identity system electronic device 1502 may exchange data with one or more of the other devices of the system 1500. In other implementations, the identity system electronic device 1502 may communicate with different devices of the system 1500 to facilitate and/or authorize direct data communication between those devices. For example, a first device may request an authorization token for data from another device and the identity system electronic device 1502 may provide that authorization token to the first device whereupon the first device may transmit the authorization token to a second device in order to authorize the second device to transmit the requested data to the first device without identity system electronic device 1502 acting as a relay.

Similarly, the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and/or the medication access system device 1503J may be any kind of any kind of electronic device and/or cloud and/or other computing arrangement and may include one more processing units, communication units, non-transitory storage media, biometric reader devices and/or other components.

Although the system 1500 is illustrated and described as including the accounting system device 1503A, scheduling system device 1503B, calendaring system device 1503C, payment system device 1503D, medical record system device 1503E, check in system device 1503F, building access system device 1503G, insurance system device 1503H, medical procedure authorization system device 1503I, and the medication access system device 1503J, it is understood that this is an example. In other implementations, any number of different systems may be included that may be operable to perform a number of different functions. Various configurations are possible and contemplated without departing from the scope of the present disclosure.

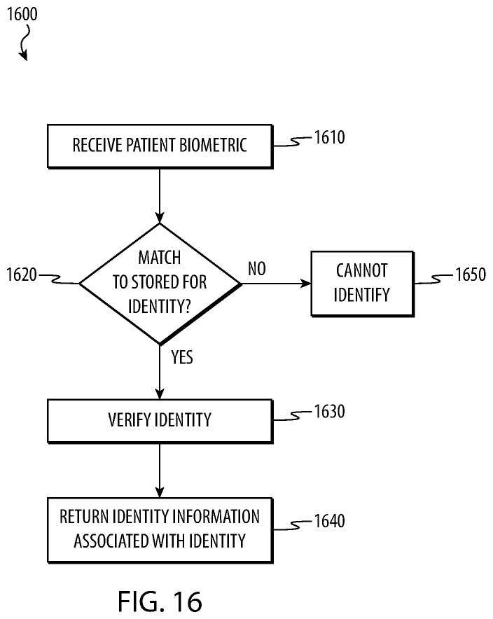

FIG. 16 is a flow chart illustrating a first example method 1600 for biometric identity system integration of medical service provider systems. This method 1600 may be performed by the systems 100, 300, 1400, 1500 of FIGS. 1, 3, 14, and/or 15.

At operation 1610, an electronic device (such as the identity system electronic devices 102, 302, 1402, 1502 of FIGS. 1, 3, 14, and/or 15) may receive a digital representation of a biometric for a patient. For example, the digital representation of the biometric may be a digital representation of a fingerprint, a blood vessel scan, a palm-vein scan, a voiceprint, a facial image, a retina image, an iris image, a deoxyribonucleic acid sequence, a heart rhythm, a gait, and so on. At operation 1620, the electronic device may determine whether or not the digital representation of the biometric matches to biometric data stored for an identity. If so, the flow may proceed to operation 1630. Otherwise, the flow may proceed to operation 1650 where the electronic device may determine that the patient cannot be identified.

At operation 1630, after the electronic device determines that the digital representation of the biometric matches to biometric data stored for an identity, the electronic device may verify the identity of the patient as the identity associated with the biometric data that matches the digital representation of the biometric. The flow may then proceed to operation 1640 where the electronic device may return identity information associated with the identity. The electronic device may return the identity information to a device that submitted the digital representation of the biometric. The identity information may include one or more names, addresses, phone numbers, medical records, insurance information, payment information, and so on.

For example, the electronic device may receive the digital representation of the biometric from a check in device that a patient accesses in a medical service provider reception area when checking in for an appointment. The electronic device may determine the patient's identity, note the patient's arrival in a scheduling system, provide payment information for the person to a payment system, arrange for medical records to be transmitted to a tablet computing device controlled by the patient's doctor, and return information to the check in device directing the patient where to go to meet the doctor. Various configurations are possible and contemplated without departing from the scope of the present disclosure.

In various examples, this example method 1600 may be implemented as a group of interrelated software modules or components that perform various functions discussed herein. These software modules or components may be executed within a cloud network and/or by one or more computing devices, such as the identity system electronic devices 102, 302, 1402, 1502 of FIGS. 1, 3, 14, and/or 15.

Although the example method 1600 is illustrated and described as including particular operations performed in a particular order, it is understood that this is an example. In various implementations, various orders of the same, similar, and/or different operations may be performed without departing from the scope of the present disclosure.

For example, the operation 1640 is illustrated and described as retuning the identity information. However, it is understood that this is an example. In some implementations, the electronic device may take an action using the identity information instead of returning the identity information. Various configurations are possible and contemplated without departing from the scope of the present disclosure.

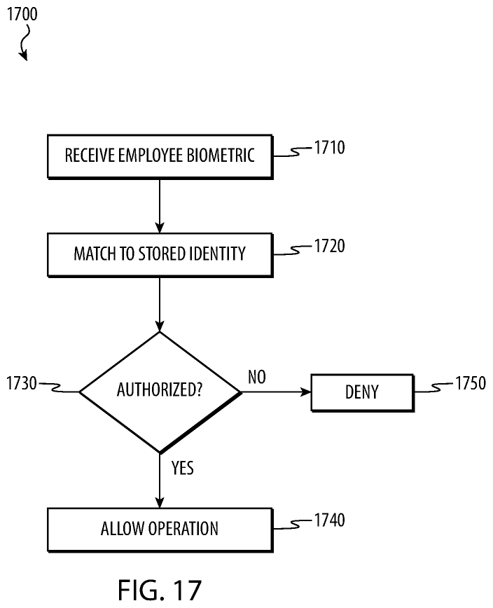

FIG. 17 is a flow chart illustrating a second example method 1700 for biometric identity system integration of medical service provider systems. This method 1700 may be performed by the systems 100, 300, 1400, 1500 of FIGS. 1, 3, 14, and/or 15.

At operation 1710, an electronic device (such as the identity system electronic devices 102, 302, 1402, 1502 of FIGS. 1, 3, 14, and/or 15) may receive a digital representation of a biometric for an employee. For example, the digital representation of the biometric may be a digital representation of a fingerprint, a blood vessel scan, a palm-vein scan, a voiceprint, a facial image, a retina image, an iris image, a deoxyribonucleic acid sequence, a heart rhythm, a gait, and so on. At operation 1720, the electronic device may match the digital representation of the biometric to biometric data stored for an identity. At 1730, the electronic device may determine whether or not the identity is authorized to perform a requested operation. If so, the flow may proceed to operation 1740. Otherwise, the flow may proceed to operation 1750 where the electronic device may deny authorization to perform the requested operation.

At operation 1740, after the electronic device determines that the identity is authorized to perform a requested operation, the electronic device may allow the operation. The operation may be accessing patient information, performing a medical service for a patient, accessing a restricted area, accessing and/or prescribing a medication, and so on.

For example, a staff member may provide a digital representation of a biometric via a front desk computing device connected to a scheduling system when attempting to schedule an appointment for a patient. The electronic device may determine the staff member's identity, determine that the staff member is authorized to access identity information for the patient, and provide the scheduling system identity information for the patient as part of allowing the staff member to schedule the appointment. Various configurations are possible and contemplated without departing from the scope of the present disclosure.

By way of another example, a medical technician may provide a digital representation of a biometric to a medication access system that controls and logs access to a medication closet. The electronic device may determine the medical technician's identity, determine that the staff member is authorized to access the medication closet, allow the access, and log the medications that the medical technician removes and for whom the medical technician removes the medications. Various configurations are possible and contemplated without departing from the scope of the present disclosure.

In various examples, this example method 1700 may be implemented as a group of interrelated software modules or components that perform various functions discussed herein. These software modules or components may be executed within a cloud network and/or by one or more computing devices, such as the identity system electronic devices 102, 302, 1402, 1502 of FIGS. 1, 3, 14, and/or 15.

Although the example method 1700 is illustrated and described as including particular operations performed in a particular order, it is understood that this is an example. In various implementations, various orders of the same, similar, and/or different operations may be performed without departing from the scope of the present disclosure.

For example, the method 1700 is illustrated and described as determining whether or not the identity is authorized to perform the operation. However, in various examples, the operation may not require authorization. In some examples, the electronic device may determine the identity in order to track the identity that performs the operation rather than determining whether or not the identity is authorized. Various configurations are possible and contemplated without departing from the scope of the present disclosure.

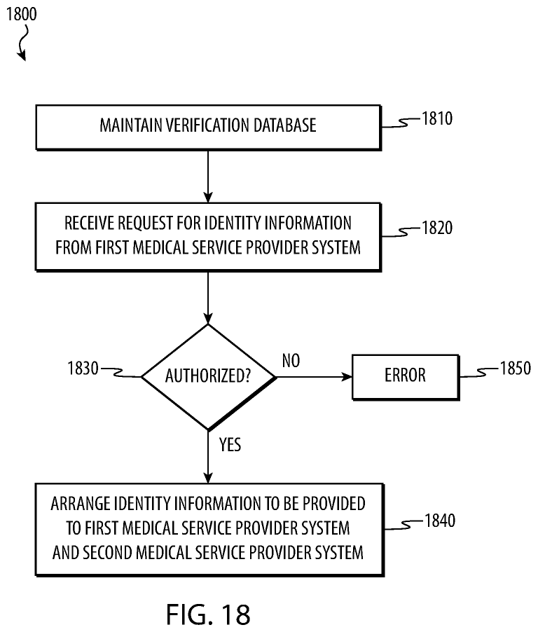

FIG. 18 is a flow chart illustrating a third example method 1800 for biometric identity system integration of medical service provider systems. This method 1800 may be performed by the systems 100, 300, 1400, 1500 of FIGS. 1, 3, 14, and/or 15.

At operation 1810, an electronic device (such as the identity system electronic devices 102, 302, 1402, 1502 of FIGS. 1, 3, 14, and/or 15) may maintain biometric data associated with identities. The electronic device may maintain biometric data associated with identities in order to verify the identities for various medical service provider and/or other systems.

At operation 1820, the electronic device may receive a request for identity information from a first medical service provider system. At operation 1830, the electronic device may determine whether or not the request is authorized. For example, the request may include a digital representation of a biometric. In some implementations of such an example, the electronic device may determine that the request is authorized when the digital representation of the biometric matches biometric data associated with an identity corresponding to the identity information. In other implementations of such an example, the electronic device may determine that the request is authorized when the digital representation of the biometric matches biometric data associated with an identity that has permission to access the identity information. If the electronic device determines that the request is authorized, the flow may proceed to 1840. Otherwise, the flow may proceed to 1850 where the electronic device may determine that an error has occurred.

At operation 1840, after the electronic device determines that the request is authorized, the electronic device may arrange for the identity information to be provided to the first medical service provider system and a second medical service provider system. Arranging for the identity information to be provided may include transmitting identity information stored by the electronic device, the electronic device obtaining the identity information from another device and transmitting the obtained identity information, instructing the device to transmit the identity information to the first and/or second medical service provider systems, and so on. The second medical service provider system may be specified in the request; may be included in a specification stored by the electronic device, in the identity information, and/or elsewhere that specifies that the identity information is to be sent to the second medical service provider system upon access by the first medical service provider system; and so on.

For example, the electronic device may receive a request for identity information of a patient from a check in device that a patient accesses in a medical service provider reception area when checking in for an appointment. The electronic device may determine that the patient's request for his own identity information is authorized, note the patient's arrival in a scheduling system, provide payment information for the person to a payment system, arrange for medical records to be transmitted to a tablet computing device controlled by the patient's doctor, and return information to the check in device directing the patient where to go to meet the doctor. Various configurations are possible and contemplated without departing from the scope of the present disclosure.

By way of another example, a staff member may request a patient's insurance information via a front desk computing device connected to a scheduling system when attempting to schedule an appointment for a patient. The electronic device may determine that the request is authorized, obtain the patient's insurance information for the scheduling system, and also provide the patient's insurance information to an insurance system that may be used to process an insurance claim for medical services provided during the appointment. Various configurations are possible and contemplated without departing from the scope of the present disclosure.

In various examples, this example method 1800 may be implemented as a group of interrelated software modules or components that perform various functions discussed herein. These software modules or components may be executed within a cloud network and/or by one or more computing devices, such as the identity system electronic devices 102, 302, 1402, 1502 of FIGS. 1, 3, 14, and/or 15.

Although the example method 1800 is illustrated and described as including particular operations performed in a particular order, it is understood that this is an example. In various implementations, various orders of the same, similar, and/or different operations may be performed without departing from the scope of the present disclosure.

For example, the operation 1850 is illustrated and described as determining that an error has occurred if the request is not authorized. However, in other examples, the electronic device may indicate that the request is not authorized and may prompt for the request to be submitted by an authorized person. Various configurations are possible and contemplated without departing from the scope of the present disclosure.

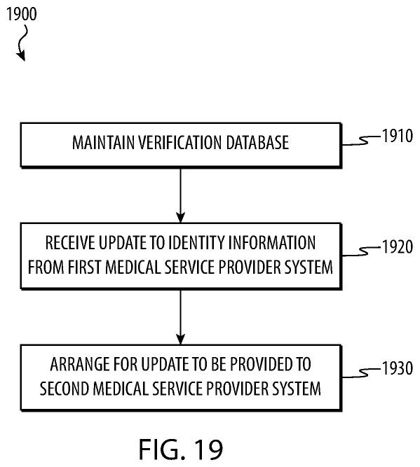

FIG. 19 is a flow chart illustrating a fourth example method 1900 for biometric identity system integration of medical service provider systems. This method 1900 may be performed by the systems 100, 300, 1400, 1500 of FIGS. 1, 3, 14, and/or 15.

At operation 1910, an electronic device (such as the identity system electronic devices 102, 302, 1402, 1502 of FIGS. 1, 3, 14, and/or 15) may maintain biometric data associated with identities, such as in order to verify the identities for one or more medical service provider and/or other systems. At operation 1920, the electronic device may receive an update to the identity information from a first medical service provider system. For example, the update may be an added and/or changed name, address, medical record, insurance information, payment information, and so on.

At 1930, the electronic device may arrange for the update to be provided to a second medical service provider system. The second medical service provider system may be specified in the request; may be included in a specification stored by the electronic device, in the identity information, and/or elsewhere that specifies that the identity information is to be sent to the second medical service provider system upon access by the first medical service provider system; and so on.

For example, the electronic device may receive a request to update identity information of a patient with new insurance information from a check in device that a patient accesses in a medical service provider reception area when checking in for an appointment. The electronic device may provide the updated insurance information to an insurance system that may be used to process an insurance claim for medical services provided during the appointment. Various configurations are possible and contemplated without departing from the scope of the present disclosure.

In various examples, this example method 1900 may be implemented as a group of interrelated software modules or components that perform various functions discussed herein. These software modules or components may be executed within a cloud network and/or by one or more computing devices, such as the identity system electronic devices 102, 302, 1402, 1502 of FIGS. 1, 3, 14, and/or 15.

Although the example method 1900 is illustrated and described as including particular operations performed in a particular order, it is understood that this is an example. In various implementations, various orders of the same, similar, and/or different operations may be performed without departing from the scope of the present disclosure.

For example, in some implementations, the method 1900 may include one or more additional operations. Such additional operations may include determining whether or not the update is authorized, updating the identity information, and so on. Various configurations are possible and contemplated without departing from the scope of the present disclosure.

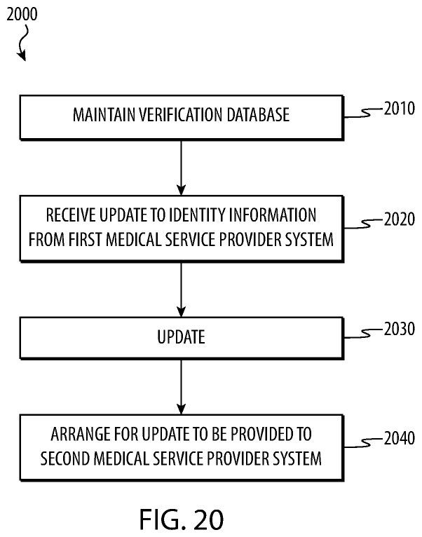

FIG. 20 is a flow chart illustrating a fifth example method 2000 for biometric identity system integration of medical service provider systems. This method 2000 may be performed by the systems 100, 300, 1400, 1500 of FIGS. 1, 3, 14, and/or 15.

At operation 2010, an electronic device (such as the identity system electronic devices 102, 302, 1402, 1502 of FIGS. 1, 3, 14, and/or 15) may maintain biometric data associated with identities, such as in order to verify the identities for one or more medical service provider and/or other systems. The biometric data may include one or more digital representations of one or more biometrics. For example, the digital representation of the biometric may include a digital representation of a fingerprint, a blood vessel scan, a palm-vein scan, a voiceprint, a facial image, a retina image, an iris image, a deoxyribonucleic acid sequence, a heart rhythm, a gait, and so on. At operation 2020, the electronic device may receive an update to the identity information from a first medical service provider system. At operation 2030, the electronic device may update the identity information. At operation 2040, the electronic device may arrange for the update to be provided to a second medical service provider system.

By way of example, a staff member may enter a new telephone number for a patient via a front desk computing device connected to a scheduling system when talking with a patient over the phone to schedule an appointment. The electronic device may update the patient's identity information with the new telephone number. The electronic device may also provide the new phone number to a medical records system that stores information about the patient. Various configurations are possible and contemplated without departing from the scope of the present disclosure.

In various examples, this example method 2000 may be implemented as a group of interrelated software modules or components that perform various functions discussed herein. These software modules or components may be executed within a cloud network and/or by one or more computing devices, such as the identity system electronic devices 102, 302, 1402, 1502 of FIGS. 1, 3, 14, and/or 15.

Although the example method 2000 is illustrated and described as including particular operations performed in a particular order, it is understood that this is an example. In various implementations, various orders of the same, similar, and/or different operations may be performed without departing from the scope of the present disclosure.

For example, the method 2000 is illustrated and described as including the operation 2010 of maintaining biometric data associated with identities. However, in other implementations, the electronic device may not maintain biometric data associated with identities. In some implementations, the electronic device may instead access biometric data associated with identities that is stored by one or more other devices. Various configurations are possible and contemplated without departing from the scope of the present disclosure.

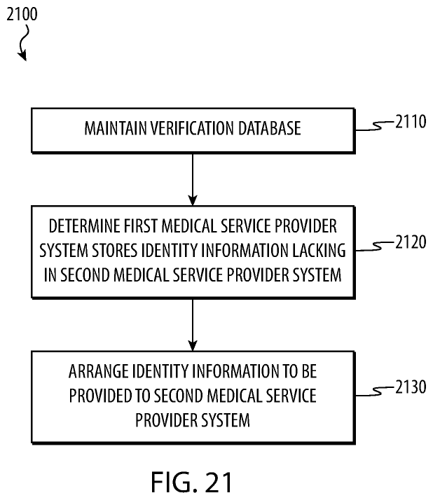

FIG. 21 is a flow chart illustrating a sixth example method 2100 for biometric identity system integration of medical service provider systems. This method 2100 may be performed by the systems 100, 300, 1400, 1500 of FIGS. 1, 3, 14, and/or 15.

At operation 2110, an electronic device (such as the identity system electronic devices 102, 302, 1402, 1502 of FIGS. 1, 3, 14, and/or 15) may maintain biometric data associated with identities, such as in order to verify the identities for one or more medical service provider and/or other systems. At operation 2120, the electronic device may determine that the first medical service provider system stores identity information that is missing from the second medical service provider system. In some implementations, the electronic device may determine that the first medical service provider system stores identity information that is missing from the second medical service provider system by searching and/or comparing indexes of data stored by the first and/or second medical service provider systems, by determining that a search has failed for data that should be present, and so on.

At operation 2130, the electronic device may arrange for the identity information to be provided to the second medical service provider system. The electronic device may obtain the identity information from the first medical service provider system and transmit the obtained identity information to the second medical service provider system, may instruct the first medical service provider system to transmit the identity information to the second medical service provider system, and so on.

For example, the electronic device may determine that a patient's telephone number is stored in a scheduling system but not a payment system. As such, the electronic device may copy the patient's telephone number from the scheduling system to the payment system. Various configurations are possible and contemplated without departing from the scope of the present disclosure.

In various examples, this example method 2100 may be implemented as a group of interrelated software modules or components that perform various functions discussed herein. These software modules or components may be executed within a cloud network and/or by one or more computing devices, such as the identity system electronic devices 102, 302, 1402, 1502 of FIGS. 1, 3, 14, and/or 15.

Although the example method 2100 is illustrated and described as including particular operations performed in a particular order, it is understood that this is an example. In various implementations, various orders of the same, similar, and/or different operations may be performed without departing from the scope of the present disclosure.

For example, the method 2100 is illustrated and described as providing identity information to a second medical service provider system that is lacking on the second medical service provider system but stored on a first medical service provider system. However, in some implementations, the first and second medical service provider systems may both store the identity information, but may store different versions. For example, one of the first and second medical service provider systems may have an updated copy of the identity information whereas the other has an outdated copy. As such, the method 2100 may replace the outdated copy with the updated copy.

By way of example, the electronic device may determine that an accounting system and an insurance system store different insurance information for a patient. The electronic device may determine which insurance information is current and arrange for the up to date insurance information to be provided to the system that has out of date insurance information so that the out of date insurance information may be replaced with the up to date insurance information. Various configurations are possible and contemplated without departing from the scope of the present disclosure.

In various implementations, a system for biometric identity system integration of medical service provider systems may include at least one non-transitory storage medium that stores instructions and at least one processor. The at least one processor may execute the instructions to maintain biometric data associated with identities in order to verify the identities for a first medical service provider system device and a second medical service provider system device; receive a request for identity information associated with an identity from the first medical service provider system device; and upon determining that the request is authorized, arrange for the identity information to be provided to the first medical service provider system device and the second medical service provider system device.

In some examples, the request may include a digital representation of a biometric. In various such examples, the identity may be a first identity and the at least one processor may determine that the request is authorized when the digital representation of the biometric matches first biometric data associated with the first identity or matches second biometric data associated with a second identity that has permission to access the identity information.

In a number of examples, the first medical service provider system device and the second medical service provider system device may be devices in at least two of an accounting system, a scheduling system, a calendaring system, a payment system, a medical record system, a check in system, a building access system, an insurance system, a medical procedure authorization system, and a medication access system. In various examples, the at least one processor may arrange for the identity information to be provided to the first medical service provider system device and the second medical service provider system device by transmitting the identity information to the first medical service provider system device and the second medical service provider system device. In some examples, the at least one processor may arrange for the identity information to be provided to the first medical service provider system device and the second medical service provider system device by instructing an electronic device to transmit the identity information to the first medical service provider system device and the second medical service provider system device. In a number or examples, the at least one processor may determine that the second medical service provider system device is authorized to receive the identity information before arranging for the identity information to be provided to the second medical service provider system device.

In some implementations, a system for biometric identity system integration of medical service provider systems may include at least one non-transitory storage medium that stores instructions and at least one processor. The at least one processor may execute the instructions to maintain biometric data associated with identities in order to verify the identities for a first medical service provider system device and a second medical service provider system device, receive an update to identity information associated with an identity from the first medical service provider system device, and arrange for the update to be provided to the second medical service provider system device.

In various examples, the update may be at least one of a name, an address, a medical record, insurance information, or payment information. In some examples, the update may be received from a person corresponding to the identity. In a number of examples, the identity may be a first identity and the update may be received from a person corresponding to a second identity that is authorized to modify the identity information.

In some examples, the at least one processor may determine that the update is authorized prior to arranging for the update to be provided to the second medical service provider system device. In a number of such examples, the at least one processor may update the identity information upon determining that the update is authorized. In various such examples, the at least one processor may determine that the update is authorized by comparing a received digital representation of a biometric to respective biometric data associated with the identity.

In a number of implementations, a system for biometric identity system integration of medical service provider systems may include at least one non-transitory storage medium that stores instructions and at least one processor. The at least one processor may execute the instructions to maintain biometric data associated with identities in order to verify the identities for a first medical service provider system device and a second medical service provider system device, determine that the first medical service provider system device stores identity information that is associated with an identity and missing from the second medical service provider system device, and arrange for the identity information to be provided to the second medical service provider system device.

In some examples, the at least one processor may receive an identity verification request including a digital representation of a biometric and provide a response to the identity verification request based on a comparison of the digital representation of the biometric to at least a portion of the biometric data. In a number of such examples, the response may identify a respective identity that is associated with respective biometric data that matches the digital representation of the biometric.

In various examples, the at least one processor may determine that the identity information is covered by a specification listing information to be duplicated on the first medical service provider system device and the second medical service provider system device. In some examples, the at least one processor may determine that the second medical service provider system device is covered by a specification listing devices on which to duplicate information from the first medical service provider system device. In a number of examples, the first medical service provider system device may not be configured to transmit the identity information directly to the second medical service provider system device absent involvement of the at least one processor.

Although the above illustrates and describes a number of embodiments, it is understood that these are examples. In various implementations, various techniques of individual embodiments may be combined without departing from the scope of the present disclosure.

As described above and illustrated in the accompanying figures, the present disclosure relates to biometric identity system integration of medical service provider systems. An identity system may communicate with different medical service provider systems in order to exchange and/or direct the exchange of information. The identity system may maintain biometric data associated with identities in order to verify the identities for the medical service provider systems. In various examples, the identity system may receive a request for identity information from a first system and arrange for the identity information to be provided to the first system and a second system upon determining that the request is authorized. In some examples, the identity system may receive an update to the identity information from the first system and arrange for the update to be provided to the second system. In a number of examples, the identity system may determine that the first system stores identity information that is missing from the second system and may arrange for the identity information to be provided to the second system.

Although the above illustrates and describes a number of embodiments, it is understood that these are examples. In various implementations, various techniques of individual embodiments may be combined without departing from the scope of the present disclosure.

The present disclosure recognizes that biometric and/or other personal data is owned by the person from whom such biometric and/or other personal data is derived. This data can be used to the benefit of those people. For example, biometric data may be used to conveniently and reliably identify and/or authenticate the identity of people, access securely stored financial and/or other information associated with the biometric data, and so on. This may allow people to avoid repeatedly providing physical identification and/or other information.

The present disclosure further recognizes that the entities who collect, analyze, store, and/or otherwise use such biometric and/or other personal data should comply with well-established privacy policies and/or privacy practices. Particularly, such entities should implement and consistently use privacy policies and practices that are generally recognized as meeting or exceeding industry or governmental requirements for maintaining security and privately maintaining biometric and/or other personal data, including the use of encryption and security methods that meets or exceeds industry or government standards. For example, biometric and/or other personal data should be collected for legitimate and reasonable uses and not shared or sold outside of those legitimate uses. Further, such collection should occur only after receiving the informed consent. Additionally, such entities should take any needed steps for safeguarding and securing access to such biometric and/or other personal data and ensuring that others with access to the biometric and/or other personal data adhere to the same privacy policies and practices. Further, such entities should certify their adherence to widely accepted privacy policies and practices by subjecting themselves to appropriate third party evaluation.

Additionally, the present disclosure recognizes that people may block the use of, storage of, and/or access to biometric and/or other personal data. Entities who typically collect, analyze, store, and/or otherwise use such biometric and/or other personal data should implement and consistently prevent any collection, analysis, storage, and/or other use of any biometric and/or other personal data blocked by the person from whom such biometric and/or other personal data is derived.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A system for biometric identity system integration of medical service provider systems, comprising:
at least one non-transitory storage medium that stores instructions; and
at least one processor that executes the instructions to:
maintain biometric data associated with identities in order to verify the identities for a first medical service provider system device and a second medical service provider system device;
receive a request for identity information associated with an identity from the first medical service provider system device; and
upon determining that the request is authorized, arrange for the identity information to be provided to the first medical service provider system device and the second medical service provider system device.

2. The system of claim 1, wherein the request includes a digital representation of a biometric.

3. The system of claim 2, wherein:
the identity is a first identity; and
the at least one processor determines that the request is authorized when the digital representation of the biometric:
matches first biometric data associated with the first identity; or
matches second biometric data associated with a second identity that has permission to access the identity information.

4. The system of claim 1, wherein the first medical service provider system device and the second medical service provider system device comprise devices in at least two of:
an accounting system;
a scheduling system;
a calendaring system;
a payment system;
a medical record system;
a check in system;
a building access system;
an insurance system;
a medical procedure authorization system; and
a medication access system.

5. The system of claim 1, wherein the at least one processor arranges for the identity information to be provided to the first medical service provider system device and the second medical service provider system device by transmitting the identity information to the first medical service provider system device and the second medical service provider system device.

6. The system of claim 1, wherein the at least one processor arranges for the identity information to be provided to the first medical service provider system device and the second medical service provider system device by instructing an electronic device to transmit the identity information to the first medical service provider system device and the second medical service provider system device.

7. The system of claim 1, wherein the at least one processor determines that the second medical service provider system device is authorized to receive the identity information before arranging for the identity information to be provided to the second medical service provider system device.

8. A system for biometric identity system integration of medical service provider systems, comprising:
at least one non-transitory storage medium that stores instructions; and
at least one processor that executes the instructions to:
maintain biometric data associated with identities in order to verify the identities for a first medical service provider system device and a second medical service provider system device;
receive an update to identity information associated with an identity from the first medical service provider system device; and
arrange for the update to be provided to the second medical service provider system device.

9. The system of claim 8, wherein the update comprises at least one of:
a name;
an address;
a medical record;
insurance information; or
payment information.

10. The system of claim 8, wherein the update is received from a person corresponding to the identity.

11. The system of claim 8, wherein:
the identity is a first identity; and
the update is received from a person corresponding to a second identity that is authorized to modify the identity information.

12. The system of claim 8, wherein the at least one processor determines that the update is authorized prior to arranging for the update to be provided to the second medical service provider system device.

13. The system of claim 12, wherein the at least one processor updates the identity information upon determining that the update is authorized.

14. The system of claim 12, wherein the at least one processor determines that the update is authorized by comparing a received digital representation of a biometric to respective biometric data associated with the identity.

15. A system for biometric identity system integration of medical service provider systems, comprising:
at least one non-transitory storage medium that stores instructions; and
at least one processor that executes the instructions to:
maintain biometric data associated with identities in order to verify the identities for a first medical service provider system device and a second medical service provider system device;
determine that the first medical service provider system device stores identity information that is associated with an identity and missing from the second medical service provider system device; and
arrange for the identity information to be provided to the second medical service provider system device.

16. The system of claim 15, wherein the at least one processor:
receives an identity verification request including a digital representation of a biometric; and
provides a response to the identity verification request based on a comparison of the digital representation of the biometric to at least a portion of the biometric data.

17. The system of claim 16, wherein the response identifies a respective identity that is associated with respective biometric data that matches the digital representation of the biometric.

18. The system of claim 15, wherein the at least one processor determines that the identity information is covered by a specification listing information to be duplicated on the first medical service provider system device and the second medical service provider system device.

19. The system of claim 15, wherein the at least one processor determines that the second medical service provider system device is covered by a specification listing devices on which to duplicate information from the first medical service provider system device.

20. The system of claim 15, wherein the first medical service provider system device is not configured to transmit the identity information directly to the second medical service provider system device absent involvement of the at least one processor.
